(19) United States
(12) Patent Application Publication
(10) Pub. No.: US 2022/0349887 A1
(43) Pub. Date: Nov. 3, 2022

(54) Compositions and Methods for Multiplex Detection of Microorganisms Using Peptide-Tagged Recombinant Infectious Agents

(71) Applicant: Laboratory Corporation of America Holdings, Burlington, NC (US)

(72) Inventors: Stephen E. Erickson, White Bear Township, MN (US); Jose S. Gil, Winnetka, CA (US); Matthew J. Brown, Burlington, NC (US)

(21) Appl. No.: 17/733,719

(22) Filed: Apr. 29, 2022

Related U.S. Application Data
(60) Provisional application No. 63/182,188, filed on Apr. 30, 2021.

(51) Int. Cl. G01N 33/569; G01N 33/58

(52) U.S. Cl. CPC G01N 33/56911; G01N 33/581

(57) ABSTRACT

Disclosed herein are methods, compositions, kits, and systems for rapid multiplex detection of a microorganism of interest. Recombinant bacteriophages encoding capture moiety-indicator protein fusion products allow for the capture of indicator proteins on a surface. Cocktail compositions of recombinant bacteriophages can be used to detect potentially harmful bacteria. The specificity of recombinant bacteriophages for binding microorganisms allows targeted and highly specific detection of a microorganism of interest.

### PRIORITY

The present application claims priority to and filing benefit of U.S. Provisional Patent Application No. 63/182,188, filed Apr. 30, 2021, which is hereby incorporated herein by reference in its entirety.

### FIELD OF THE INVENTION

This disclosure relates to compositions, methods, and systems for the detection of microorganisms using infectious agents.

### BACKGROUND

There is a strong interest in improving speed and sensitivity for detection of bacteria, viruses, and other microorganisms in biological, food, water, and clinical samples. Microbial pathogens can cause substantial morbidity among humans and domestic animals, as well as immense economic loss. Also, detection of microorganisms is a high priority for the Food and Drug Administration (FDA) and Centers for Disease Control (CDC), as well as the United States Department of Agriculture (USDA), given outbreaks of life-threatening or fatal illness caused by ingestion of food contaminated with certain microorganisms, e.g., Escherichia coli, Cronobacter spp., Salmonella spp., Listeria spp., or Staphylococcus spp.

Traditional microbiological tests for the detection of bacteria rely on non-selective and selective enrichment cultures followed by plating on selective media and further testing to confirm suspect colonies. Such procedures can require several days. A variety of rapid methods have been investigated and introduced into practice to reduce the time requirement. However, these methods have drawbacks. For example, techniques involving direct immunoassays or gene probes generally require an overnight enrichment step in order to obtain adequate sensitivity. Polymerase chain reaction (PCR) tests also include an amplification step and therefore are capable of both very high sensitivity and selectivity; however, the sample size that can be economically subjected to PCR testing is limited. With dilute bacterial suspensions, most small subsamples will be free of cells and therefore purification and/or lengthy enrichment steps are still required.

The time required for traditional biological enrichment is dictated by the growth rate of the target bacterial population of the sample, by the effect of the sample matrix, and by the required sensitivity. In practice, most high sensitivity methods employ an overnight incubation and take about 24 hours overall. Due to the time required for cultivation, these methods can take up to three days, depending upon the organism to be identified and the source of the sample. This lag time is generally unsuitable as the contaminated food, water, or other product may have already made its way into livestock or humans. In addition, increases in antibiotic-resistant bacteria and biodefense considerations make rapid identification of bacterial pathogens in water, food and clinical samples critical priorities worldwide.

Therefore, there is a need for more rapid, simple, and sensitive detection and identification of microorganisms, such as bacteria and other potentially pathogenic microorganisms.

### SUMMARY

Embodiments of the disclosure comprise compositions, methods, systems, and kits for the detection of microorganisms. The disclosure may be embodied in a variety of ways.

In a first aspect of the present disclosure is a recombinant bacteriophage comprising a genetic construct inserted into the bacteriophage genome, wherein the genetic construct comprises an indicator gene and a capture moiety. One embodiment of the present disclosure is an indicator cocktail composition comprising at least two recombinant bacteriophages, wherein the recombinant bacteriophages each comprise a genetic construct inserted into the bacteriophage genome, wherein the genetic construct comprises an indicator gene and a capture moiety. The capture moiety may be a peptide or protein tag. Another embodiment is a multiplex cocktail composition comprising at least two indicator cocktail compositions, wherein the first indicator cocktail composition is specific for a first bacteria of interest and a second cocktail composition is specific for a second bacteria of interest.

In a second aspect of the present disclosure is a method of detecting a bacteria of interest in a sample comprising the steps of: (i) obtaining the sample; (ii) incubating the sample with at least one indicator cocktail composition, wherein the recombinant bacteriophage comprises a genetic construct inserted into the bacteriophage genome, and wherein the genetic construct comprises an indicator gene encoding an indicator protein product; (iii) capturing the indicator protein product; and (iv) detecting the captured indicator protein product, wherein detection of the captured indicator protein product indicates the bacteria of interest is present in the sample.

In a third aspect of the disclosure is a method of detecting a bacteria of interest in a sample comprising the steps of: (i) obtaining the sample; (ii) incubating the sample with at least one indicator cocktail composition, wherein the recombinant bacteriophage comprises a genetic construct inserted into the bacteriophage genome, and wherein the genetic construct comprises an indicator gene and a capture moiety encoding a capture moiety-indicator protein fusion product; (iii) capturing the capture moiety-indicator protein fusion product; and (iv) detecting the captured indicator protein product, wherein detection of the captured indicator protein product indicates the bacteria of interest is present in the sample.

In a fourth aspect of the disclosure is a kit for detecting a bacteria of interest comprising: (i) recombinant bacteriophage comprising a genetic construct inserted into the bacteriophage genome, wherein the recombinant bacteriophage comprises a genetic construct inserted into the bacteriophage genome, and wherein the genetic construct comprises an indicator gene and a capture moiety encoding a capture moiety-indicator protein fusion product; and (ii) a surface for capturing the capture moiety-indicator protein fusion product, wherein the surface comprises a binding partner. The capture moiety can be a peptide or protein tag.

In a fifth aspect of the disclosure is a system for detecting a bacteria of interest comprising: (a) a recombinant bacteriophage comprising a genetic construct inserted into the bacteriophage genome, wherein the recombinant bacteriophage comprises a genetic construct inserted into the bacteriophage genome, and wherein the genetic construct comprises an indicator gene and a capture moiety encoding a capture moiety-indicator protein fusion product; (b) a surface for capturing capture moiety-indicator protein fusion product, wherein the surface comprises a binding partner; and (c) a component for detecting the indicator protein product.

### BRIEF DESCRIPTION OF THE FIGURES

The present disclosure may be better understood by referring to the following non-limiting figures.

FIG. 1 depicts homologous recombination inserts according to an embodiment of the invention illustrating a genetic construct comprising a luciferase gene with either an N-terminal or C-terminal peptide tag, a bacteriophage late gene promoter, and a ribosomal binding site (RBS). The promoter depicted is in addition to and separate from the endogenous late gene promoter upstream of the endogenous late genes, such as the gene for major capsid protein (MCP).

FIG. 2 depicts one embodiment of the methods disclosed herein, showing a flow diagram of a multiplex lateral flow strip assay for detection of Enterococcus faecium, Staphylococcus aureus, Klebsiella pneumoniae, Acinetobacter baumannii, Pseudomonas aeruginosa and Enterobacter species (ESKAPE pathogens) in whole blood using recombinant bacteriophages.

FIG. 3 depicts one embodiment of the methods disclosed herein, showing a flow diagram of a multiplex assay using a microtiter plate for detection of ESKAPE pathogens in whole blood using recombinant bacteriophages.

### DETAILED DESCRIPTION OF THE INVENTION

Disclosed herein are compositions, methods, kits, and systems that demonstrate surprising sensitivity for multiplex detection of bacteria of interest, in a variety of test samples (e.g., biological, food, water, surface, and environmental). The present disclosure describes the use of genetically modified infectious agents in multiplex assays.

Detection can be achieved in a shorter timeframe than previously thought possible using genetically modified infectious agents in assays performed without culturing for enrichment, or in some embodiments with minimal enrichment periods. Also surprising is the success of using a potentially high multiplicity of infection (MOI), or high concentrations of plaque forming units (PFU), for incubation with a test sample. Such high phage concentrations (PFU/mL) were previously purported to be detrimental in bacterium detection assays, as they were purported to cause “lysis from without.” However, a high concentration of phage can facilitate finding, binding, and infecting a low number of target cells.

The compositions, methods, systems, and kits of the disclosure may comprise infectious agents for use in detection of microorganisms such as Enterococcus faecium, Staphylococcus aureus, Klebsiella pneumoniae, Acinetobacter baumanni, Pseudomonas aeruginosa, Salmonella spp., Cronobacter spp., Campylobacter spp., Listeria spp., Enterobacter spp, and E. coli. In one aspect of the present disclosure, a recombinant indicator bacteriophage comprises a genetic construct. In certain instances, the genetic construct is inserted into a wild-type bacteriophage genome. In certain embodiments, the genetic construct may be inserted into a late gene (i.e., class III) region of the bacteriophage. In some embodiments, the genetic construct comprises an indicator gene. In some embodiments, the indicator gene encodes an indicator protein. In some instances, the genetic construct further comprises a capture moiety. In some embodiments, the genetic construct comprises genes for a fused indicator protein-capture moiety protein.

In some embodiments, the disclosure comprises a method for detecting a particular bacteria of interest in a sample comprising the steps of incubating the sample with a recombinant indicator bacteriophage comprising an indicator gene and a capture moiety, wherein the indicator gene encodes an indicator protein. If the bacteria of interest are present in the sample, the recombinant bacteriophages will infect the bacteria, thereby expressing the indicator gene. Thus, in some embodiments, the method comprises treatment with lysis buffer to lyse the bacteria infected with the recombinant bacteriophages prior to capturing the indicator protein product or the capture moiety-indicator protein product fusion. In some embodiments the capture moiety is a peptide or protein tag.

Thus, some embodiments of the present disclosure solve a need by using bacteriophage-based methods for amplifying a detectable signal indicating the presence of bacteria. In certain embodiments as little as a single bacterium is detected. The principles applied herein can be applied to the detection of a variety of microorganisms. Because of numerous binding sites for an infectious agent on the surface of a microorganism, the capacity to produce one hundred or more agent progeny during infection, and the potential for high level expression of an encoded indicator moiety, the infectious agent or an indicator moiety can be more readily detectable than the microorganism itself. In this way, embodiments of the present disclosure can achieve tremendous signal amplification from even a single infected cell.

Aspects of the present disclosure utilize the high specificity of binding agents that can bind to particular microorganisms, such as the binding component of infectious agents, as a means to detect and/or quantify the specific microorganism in a sample. In some embodiments, the present disclosure utilizes the high specificity of infectious agents such as bacteriophage.

Embodiments of the methods and systems of the disclosure can be applied to detection and quantification of a variety of microorganisms (e.g., bacteria) in a variety of circumstances, including but not limited to detection of pathogens from clinical, veterinary, environmental, food, water, and commercial samples. The methods of the present disclosure provide high detection sensitivity and specificity rapidly. In some embodiments detection is possible within a single replication cycle of the bacteriophage, which is unexpected.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well known in the art and as described in various general and more specific references that are discussed throughout the present specification unless otherwise indicated. Enzymatic reactions and purification techniques are performed according to manufacturer's specifications, as commonly accomplished in the art or as described herein. The nomenclatures used in connection with the laboratory procedures and techniques described herein are those well-known and commonly used in the art.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

The following terms, unless otherwise indicated, shall be understood to have the following meanings:

As used herein, the terms “a”, “an”, and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among samples.

As used herein, “solid support” or “support” means a structure that provides a substrate and/or surface onto which biomolecules may be bound. For example, a solid support may be an assay well (i.e., such as a microtiter plate or multi-well plate), or the solid support may be a location on a filter, an array, or a mobile support, such as a bead or a membrane (e.g., a filter plate, latex particles, paramagnetic particles, or lateral flow strip).

As used herein, “binding agent” or “binding partner” refers to a molecule that can specifically and selectively bind to a second (i.e., different) molecule of interest. The interaction may be non-covalent, for example, as a result of hydrogen bonding, van der Waals interactions, or electrostatic or hydrophobic interactions, or it may be covalent. The term “soluble binding agent” refers to a binding agent that is not associated with (i.e., covalently or non-covalently bound) to a solid support. The term “immobilized binding partner” refers to a binding agent that is associated with (i.e., covalently or non-covalently bound) to a solid support.

As used herein, the term “bioluminescence” refers to production and emission of light by a chemical reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

As used herein the term “complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance to form a complex (e.g., from interaction elements), for example, to place the elements in the proper conformation for complementarity, to co-localize complementary elements, to lower interaction energy for complementary, etc.

As used herein, the term “complex” refers to an assemblage or aggregate of molecules (e.g., peptides, polypeptides, proteins, etc.) in direct and/or indirect contact with one another. In one aspect, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., a peptide and polypeptide) is formed under assay conditions such that the complex is thermodynamically favored (e.g., compared to a non-aggregated, or non-complexed, state of its component molecules). As used herein the term “complex,” unless described as otherwise, refers to the assemblage of two or more molecules (e.g., peptides, polypeptides, proteins, or a combination thereof).

As used herein, the term “non-luminescent” refers to an entity (e.g., peptide, polypeptide, complex, protein, etc.) that exhibits the characteristic of not emitting a detectable amount of light in the visible spectrum (e.g., in the presence of a substrate). For example, an entity may be referred to as non-luminescent if it does not exhibit detectable luminescence in a given assay. As used herein, the term “non-luminescent” is synonymous with the term “substantially non-luminescent. For example, a non-luminescent polypeptide is substantially non-luminescent, exhibiting, for example, a 10-fold or more (e.g., 100-fold, 200-fold, 500-fold, 1×10³-fold, 1×10⁴-fold, 1×10⁵-fold, 1×10⁶-fold, 1×10⁷-fold, etc.) reduction in luminescence compared to a complex of the non-luminescent polypeptide with its non-luminescent complement peptide. In some embodiments, an entity is “non-luminescent” if any light emission is sufficiently minimal so as not to create interfering background for a particular assay.

As used herein, the terms “non-luminescent peptide” and “non-luminescent polypeptide” refer to peptides and polypeptides that exhibit substantially no luminescence (e.g., in the presence of a substrate), or an amount that is beneath the noise, or a 10-fold or more (e.g., 100-fold, 200-fold, 500-fold, 1×10³-fold, 1×10⁴-fold, 1×10⁵-fold, 1×10⁶-fold, 1×10⁷-fold, etc.) when compared to a significant signal (e.g., luminescent complex) under standard conditions (e.g., physiological conditions, assay conditions, etc.) and with typical instrumentation (e.g., luminometer, etc.). In some embodiments, such non-luminescent peptides and polypeptides assemble, according to the criteria described herein, to form a bioluminescent complex. As used herein, a “non-luminescent element” is a non-luminescent peptide or non-luminescent polypeptide. The term “bioluminescent complex” refers to the assembled complex of two or more non-luminescent peptides and/or non-luminescent polypeptides. The bioluminescent complex catalyzes or enables the conversion of a substrate for the bioluminescent complex into an unstable form; the substrate subsequently emits light. When uncomplexed, two non-luminescent elements that form a bioluminescent complex may be referred to as a “non-luminescent pair.” If a bioluminescent complex is formed by three or more non-luminescent peptides and/or non-luminescent polypeptides, the uncomplexed constituents of the bioluminescent complex may be referred to as a “non-luminescent group.”

As used herein, an “analyte” refers to a molecule, compound or cell that is being measured. The analyte of interest may, in certain embodiments, interact with a binding agent. As described herein, the term “analyte” may refer to a protein or peptide of interest. An analyte may be an agonist, an antagonist, or a modulator. Or, an analyte may not have a biological effect. Analytes may include small molecules, sugars, oligosaccharides, lipids, peptides, peptidomimetics, organic compounds and the like.

As used herein, “detectable moiety” or “detectable biomolecule” or “reporter” or “indicator” or “indicator moiety” refers to a molecule that can be measured in a quantitative assay. For example, an indicator moiety may comprise an enzyme that may be used to convert a substrate to a product that can be measured. An indicator moiety may be an enzyme that catalyzes a reaction that generates bioluminescent emissions (e.g., luciferase). Or, an indicator moiety may be a radioisotope that can be quantified. Or, an indicator moiety may be a fluorophore. Or, other detectable molecules may be used.

As used herein, “bacteriophage” or “phage” includes one or more of a plurality of bacterial viruses. In this disclosure, the terms “bacteriophage” and “phage” include viruses such as mycobacteriophage (such as for TB and paraTB), mycophage (such as for fungi), mycoplasma phage, and any other term that refers to a virus that can invade living bacteria, fungi, mycoplasma, protozoa, yeasts, and other microscopic living organisms and uses them to replicate itself. Here, “microscopic” means that the largest dimension is one millimeter or less. Bacteriophages are viruses that have evolved in nature to use bacteria as a means of replicating themselves. A phage does this by attaching itself to a bacterium and injecting its DNA (or RNA) into that bacterium, and inducing it to replicate the phage hundreds or even thousands of times. This is referred to as phage amplification.

As used herein, “late gene region” refers to a region of a viral genome that is transcribed late in the viral life cycle. The late gene region typically includes the most abundantly expressed genes (e.g., structural proteins assembled into the bacteriophage particle). Late genes are synonymous with class III genes and include genes with structure and assembly functions. For example, the late genes (synonymous with class III,) are transcribed in phage T7, e.g., from 8 minutes after infection until lysis, class I (e.g., RNA polymerase) is early from 4-8 minutes, and class II from 6-15 minutes, so there is overlap in timing of II and III. A late promoter is one that is naturally located and active in such a late gene region.

As used herein, “culturing for enrichment” refers to traditional culturing, such as incubation in media favorable to propagation of microorganisms, and should not be confused with other possible uses of the word “enrichment,” such as enrichment by removing the liquid component of a sample to concentrate the microorganism contained therein, or other forms of enrichment that do not include traditional facilitation of microorganism propagation. Culturing for enrichment for periods of time may be employed in some embodiments of methods described herein.

As used herein “recombinant” refers to genetic (i.e., nucleic acid) modifications as usually performed in a laboratory to bring together genetic material that would not otherwise be found. This term is used interchangeably with the term “modified” herein.

As used herein “RLU” refers to relative light units as measured by a luminometer (e.g., GLOMAX® 96) or similar instrument that detects light. For example, the detection of the reaction between luciferase and appropriate substrate (e.g., NANOLUC® with NANO-GLO®) is often reported in RLU detected.

As used herein “time to results” refers to the total amount of time from beginning of sample incubation to generated result. Time to results does not include any confirmatory testing time. Data collection can be done at any time after a result has been generated.

As used herein “reporter gene” or “indicator gene” may refer to a complete gene or to a portion of a gene. For example, the use of indicator gene or reporter gene herein may include a nucleotide sequence that encodes a smaller peptide subunit, i.e., that is transcribed and translated into a partial protein.

As used herein, “capture moiety” refers to any compound or moiety that can be used to capture a target. For example, any of the following can be used as a capture moiety: Albumin-binding protein (ABP), Alkaline Phosphatase (AP), AUI epitope, AUS epitope, Bacteriophage T7 epitope (T7-tag), Bacteriophage V5 epitope (VS-tag), Biotin-carboxy carrier protein (BCCP), Bluetongue virus tag (B-tag), Calmodulin binding peptide (CBP), Chloramphenicol Acetyl Transferase (CAT), Cellulose binding domain (CBP), Chitin binding; domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione S-transferase (GST), Human influenza hemagglutinin (HA), HaloTag®, Histidine affinity tag (HAT), Horsera.dish Peroxidase (HRP), HSV epitope, Ketosteroid isomerase (KSI), KT3 epitope, LacZ, Luciferase, Maltose-binding protein (MBP), Myc epitope, NusA, PDZ domain, PDZ ligand, Polyarginine (Arg-tag), Polyaspartate (Asp-tag), Polycysteine (Cys-tag), Polyhistidine (His-tag), Polyphenylalanine (Phe-tag), Profinity eXact, Protein C, S1-tag, S-tag, Streptavadin-binding peptide (SBP), Staphylococcal protein A (Protein A), Staphylococcal protein G (Protein G), Strep-tag, Streptavadin, Small Ubiquitin-like Modifier (SUMO), Tandem Affinity Purification (TAP), T7 epitope, Thioredoxin (Trx), TrpE, Ubiquitin, Universal, or VSV-G.

As used herein, “construct” or “genetic construct” refers to genetic material that can be added (e.g., via recombinant techniques) to a bacteriophage genome to generate a recombinant bacteriophage.

### Samples

Each of the embodiments of the compositions, methods, kits, and systems of the disclosure can allow for the rapid and sensitive detection of microorganisms. For example, methods according to the present disclosure can be performed in a shortened time period with superior results. Microorganisms of interest detectable by the compositions, methods, kits, and systems disclosed herein include, but are not limited to, bacteria or mycobacteria.

Microbes detected by the methods and systems of the present invention include pathogens that are of natural, commercial, medical, or veterinary concern. Such pathogens include Gram-negative bacteria, Gram-positive bacteria, and mycoplasmas. Any microbe for which an infectious agent that is specific for the particular microbe has been identified can be detected by the methods of the present invention. Those skilled in the art will appreciate that there is no limit to the application of the present methods other than the availability of the necessary specific infectious agent/microbe pairs.

The sample may be medical, veterinary, pharmaceutical, clinical, laboratory, food, environmental, or water samples, or any combination thereof. Medical and veterinary samples may be biological samples, for example urine, nasal, mucus, saliva, blood, sputum, cerebrospinal fluid, and fecal samples. In certain embodiments, the blood sample may be whole blood, serum, or plasma. Medical and veterinary samples may be obtained from a human or an animal.

Food samples may be of meat, poultry, processed foods, milk, cheese, other dairy products, beverages, and produce rinsates. Food samples may be cooked or uncooked. Water samples may be obtained from any water source, including environmental water sources, drinking water, and bottled water. Environmental samples may be obtained from surfaces (e.g., floors, walls, ceilings, air filters, equipment) of industrial plants, food preparation or processing areas, medical facilities, laboratories, and veterinary facilities or soil sources and water sources.

In certain embodiments, the sample is obtained from a surface. The surface may comprise a portion of any equipment, instrument, or device, including but not limited to medical devices, laboratory equipment, food processing equipment, and commercial surfaces.

Medical devices include, but are not limited to medical implants, medical laboratory equipment, surgical instruments, general examination equipment, medical electronic equipment, medical optical equipment, instruments and endoscopic equipment, medical laser equipment, high-frequency medical equipment, equipment and appliances for operating room and consulting room, and dental equipment and apparatuses.

In certain embodiments, a food processing surface is any surface that comes into contact with food whether through manufacturing or food-handling. Food processing equipment refers to the components, processing machines, and systems used to handle, prepare, cook, store, and package food and food products. A food processing surface includes, but is not limited to a surface of a tool, a machine, equipment, or structure that is employed as part of a food production, processing, preparation, or storage activity. Examples of food processing surfaces include surfaces of food processing or preparation equipment and of floors, walls, or fixtures of structures in which food processing occurs.

Samples may be liquid, solid, or semi-solid. In some embodiments, samples may be obtained from a surface. For example, samples may be obtained from a solid surface by swabbing, rinsing, irrigating, washing, cleaning, or rinsing the surface. In some embodiments, samples may be swabs of solid surfaces (e.g., medical devices or food processing equipment). In other embodiments, irrigation may be used to collect the sample. Irrigation is the flow of a solution (e.g., saline) across a surface. Thus in some embodiments, the sample is a surface irrigant.

In some embodiments, samples may be used directly in the detection methods of the present disclosure, without preparation, concentration, dilution, purification, or isolation. For example, liquid samples, including but not limited to, biological fluids (e.g., whole blood), surface irrigants, water, milk, and juices may be assayed directly. Samples may be diluted or suspended in solution, which may include, but is not limited to, a buffered solution or a bacterial culture medium. In some embodiments, samples may be used directly in the detection methods of the present disclosure, without preparation, concentration, or dilution. For example, liquid samples, including but not limited to, milk and juices, may be assayed directly. Samples may be diluted or suspended in a solution, which may include, but is not limited to, a buffered solution or a bacterial culture medium. A sample that is a solid or semi-solid may be suspended in a liquid by mincing, mixing or macerating the solid in the liquid. A sample should be maintained within a pH range that promotes bacteriophage attachment to the host bacterial cell. A sample should also contain the appropriate concentrations of divalent and monovalent cations, including but not limited to Na⁺, Mg⁺, and Ca⁺. Preferably a sample is maintained at a temperature that maintains the viability of any pathogen cells contained within the sample.

In some embodiments of the detection assay, the sample is maintained at a temperature that maintains the viability of any pathogen cell present in the sample. For example, during steps in which bacteriophages are attaching to bacterial cells, it is preferable to maintain the sample at a temperature that facilitates bacteriophage attachment. During steps in which bacteriophages are replicating within an infected bacterial cell or lysing such an infected cell, it is preferable to maintain the sample at a temperature that promotes bacteriophage replication and lysis of the host. Such temperatures are at least about 25 degrees Celsius (C), more preferably no greater than about 45 degrees C., most preferably about 37 degrees C. In some instances, the samples may be subjected to gentle mixing or shaking during bacteriophage attachment, replication and cell lysis.

Detecting the presence of microorganisms is important across several industries. For example, detection of microorganisms on a medical surface is important in the prevention of healthcare associated infections (HAIs). Similarly, detection of microorganisms on a food processing surface is important in the prevention of food-borne illnesses. Contamination of surfaces may occur as a result of inadequate cleaning, improper selection of a disinfecting agent, failure to follow recommended cleaning, disinfection, and/or sterilization procedures, and inability to use sterilization processes.

Bacterial cells detectable by the present disclosure include, but are not limited to, highly pathogenic bacteria. For example, all species of Salmonella, all strains of Escherichia coli, Cronobacter, Staphylococcus, all species of Listeria, including, but not limited to L. monocytogenes, and all species of Campylobacter. Bacterial cells detectable by the present invention include, but are not limited to, bacterial cells that are pathogens of medical or veterinary significance. Such pathogens include, but are not limited to, Bacillus spp., Acinetobacter baumanii, Enterococcus faecium, Shigella flexneri, Bordetella pertussis, Camplyobacter jejuni, Chlamydia pneumoniae, Clostridium perfringens, Enterobacter spp., Klebsiella pneumoniae, Mycoplasma pneumoniae, Salmonella typhi, Shigella sonnei, Staphylococcus aureus, and Streptococcus spp. The emergence of drug resistance among bacterial pathogens presents significant challenges for treatment of infection. Thus, in certain embodiments, bacterial cells detectable by the present invention include antibiotic-resistant bacteria (e.g., methicillin-resistant Staphylococcus aureus (MRSA)). Bacterial cells detectable by the present disclosure include, but are not limited to, bacterial cells that are food or water borne pathogens. Exemplary food-borne pathogens include E. coli, Salmonella spp., and Listeria spp. Water-borne pathogens include E. coli , Salmonella spp., Campylobacter spp., Mycobacterium spp.

Additional bacterial cells detectable by the present disclosure include, but are not limited to, all species of Staphylococcus, including, but not limited to S. aureus, Salmonella spp., Klebsiella spp. Pseudomonas spp., Streptococcus spp., all strains of Escherichia coli, Listeria spp., including, but not limited to L. monocytogenes, Campylobacter spp., Bacillus spp., Bordetella pertussis, Campylobacter jejuni, Chlamydia pneumoniae, Clostridium perfringens, Enterobacter spp., Klebsiella pneumoniae, Mycoplasma pneumoniae, Salmonella typhi, Shigella sonnei, and Streptococcus spp. In some embodiments, bacterial cells detectable by the present disclosure are those that are known causative agents of infection.

Additional microorganisms the antibiotic resistance of which can be determined using the claimed compositions, methods, kits, and systems include but are not limited to Abiotrophia adiacens, Acinetobacter baumanii, Actinomycetaceae, Bacteroides, Cytophaga and Flexibacter phylum, Bacteroides fragilis, Bordetella pertussis, Bordetella spp., Campylobacter jejuni and C. coli, Candida albicans, Candida dubliniensis, Candida glabrata, Candida guilliermondii, Candida krusei, Candida lusitaniae, Candida parapsilosis, Candida tropicalis, Candida zeylanoides, Candida spp., Chlamydia pneumoniae, Chlamydia trachomatis, Clostridium spp., Corynebacterium spp., Cronobacter spp, Crypococcus neoformans, Cryptococcus spp., Cryptosporidium parvum, Entamoeba spp., Enterobacteriaceae group, Enterococcus casseliflavus-flavescens-gallinarum group, Enterococcus faecalis, Enterococcus faecium, Enterococcus gallinarum, Enterococcus spp., Escherichia coli and Shigella spp. group, Gemella spp., Giardia spp., Haemophilus influenzae, Klebsiella pneumoniae, Legionella pneumophila, Legionella spp., Leishmania spp., Mycobacteriaceae family, Mycoplasma pneumoniae, Neisseria gonorrhoeae, Pseudomonas aeruginosa, Pseudomonads group, Staphylococcus aureus, Staphylococcus epidermidis, Staphylococcus haemolyticus, Staphylococcus hominis, Staphylococcus saprophyticus, Staphylococcus spp., Streptococcus agalactiae, Streptococcus pneumoniae, Streptococcus pyogenes, and Streptococcus spp.

In some instances, the compositions, methods, kits, and systems described herein are capable of detecting Shiga toxin-producing E. coli (STEC) infections. E. coli O157:H7 is the most common strain of STEC; however, several other strains of E. coli are also capable of producing Shiga toxin. Thus, in some embodiments, the compositions, methods, kits, and systems described herein are capable of detecting multiple strains of E. coli in one sample. In certain instances, the compositions, methods, kits, and systems described herein are capable of detecting STEC strains while excluding non-Shiga toxin producing strains of E. coli.

In some embodiments, the compositions, methods, kits, and systems described herein are capable of detecting ESKAPE pathogens. ESKAPE pathogens are a group of pathogens that commonly cause hospital-acquired infections with drug resistant properties. The six ESKAPE pathogens are Enterococcus faecium, Staphylococcus aureus, Klebsiella pneumoniae, Acinetobacter baumannii, Pseudomonas aeruginosa and Enterobacter species.

### Indicator Recombinant Bacteriophage

As described in detail herein, the compositions, methods, systems, and kits of the disclosure may comprise infectious agents for use in detection of microorganisms. In certain embodiments, the disclosure may include a composition comprising a recombinant indicator bacteriophage, wherein the bacteriophage genome is genetically modified to include a genetic construct comprising an indicator gene. The compositions of the disclosure may be embodied in a variety of ways.

In some embodiments, a recombinant bacteriophage is constructed from a bacteriophage specific for a particular bacteria of interest. Thus, in some embodiments, the recombinant indicator phage is constructed from a wild-type bacteriophage specific for one of the following bacteria: all species of Staphylococcus, including, but not limited to S. aureus, Salmonella spp., Klebsiella spp. Pseudomonas spp., Streptococcus spp., all strains of Escherichia coli, Listeria spp., including, but not limited toL. monocytogenes, Campylobacter spp., Bacillus spp., Bordetella pertussis, Campylobacter jejuni, Chlamydia pneumoniae, Clostridium perfringens, Enterobacter spp., Klebsiella pneumoniae, Mycoplasma pneumoniae, Salmonella typhi, Shigella sonnei, and Streptococcus spp. In some embodiments, the recombinant bacteriophages described herein are constructed from wild-type bacteriophage specific to bacteria that are known causative agents of infection.

In some embodiments, the recombinant bacteriophages are constructed from wild-type bacteriophages specific for an antibiotic resistant microorganism. For example, ESKAPE pathogens are a group of pathogens with drug resistant properties that commonly cause hospital-acquired infections. The six ESKAPE pathogens are Enterococcus faecium, Staphylococcus aureus, Klebsiella pneumoniae, Acinetobacter baumannii, Pseudomonas aeruginosa and Enterobacter species. Thus, in some embodiments, the recombinant bacteriophages are constructed from wild-type bacteriophages specific for at least one ESKAPE pathogen. In some embodiments, the recombinant bacteriophages are constructed from wild-type bacteriophages specific for STEC strains but are not specific for non-Shiga toxin producing strains of E. coli.

Additional wild-type bacteriophages that may be used in the compositions, methods, kits, and systems described herein are specific for antibiotic resistant bacteria, including but not limited to Abiotrophia adiacens, Acinetobacter baumanii, Actinomycetaceae, Bacteroides, Cytophaga and Flexibacter phylum, Bacteroides fragilis, Bordetella pertussis, Bordetella spp., Campylobacter jejuni and C. coli, Candida albicans, Candida dubliniensis, Candida glabrata, Candida guilliermondii, Candida krusei, Candida lusitaniae, Candida parapsilosis, Candida tropicalis, Candida zeylanoides, Candida spp., Chlamydia pneumoniae, Chlamydia trachomatis, Clostridium spp., Corynebacterium spp., Cronobacter spp, Crypococcus neoformans, Cryptococcus spp., Cryptosporidium parvum, Entamoeba spp., Enterobacteriaceae group, Enterococcus casseliflavus-flavescens-gallinarum group, Enterococcus faecalis, Enterococcus faecium, Enterococcus gallinarum, Enterococcus spp., Escherichia coli and Shigella spp. group, Gemella spp., Giardia spp., Haemophilus influenzae, Klebsiella pneumoniae, Legionella pneumophila, Legionella spp., Leishmania spp., Mycobacteriaceae family, Mycoplasma pneumoniae, Neisseria gonorrhoeae, Pseudomonas aeruginosa, Pseudomonads group, Staphylococcus aureus, Staphylococcus epidermidis, Staphylococcus haemolyticus, Staphylococcus hominis, Staphylococcus saprophyticus, Staphylococcus spp., Streptococcus agalactiae, Streptococcus pneumoniae, Streptococcus pyogenes, and Streptococcus spp.

As discussed herein, such phage may replicate inside of the bacteria to generate hundreds of progeny phage. Detection of the indicator gene inserted into the phage can be used as a measure of the bacteria in the sample. The wild-type bacteriophages used with the compositions, methods, kits, and systems described herein may be selected based off specificity for the bacteria of interest.

In some embodiments, the selected wild-type bacteriophage is from the Caudovirales order of phages. Caudovirales are an order of tailed bacteriophages with double-stranded DNA (dsDNA) genomes. Each virion of the Caudovirales order has an icosahedral head that contains the viral genome and a flexible tail. The Caudovirales order comprises five bacteriophage families: Myoviridae (long contractile tails), Siphoviridae (long non-contractile tails), Podoviridae (short non-contractile tails), Ackermannviridae, and Herelleviridae. The term myovirus can be used to describe any bacteriophage with an icosahedral head and a long contractile tail, which encompasses bacteriophages within both the Myoviridae Ackermannviridae, and Herelleviridae families.

In some embodiments, the selected wild-type phages is specific for one or more Listeria spp. In some embodiments, the selected wild-type bacteriophage is a member of the Myoviridae family such as, Listeria phage B054. T4 and T4likevirus (aka tequatrovirus). In other embodiments, the selected wild-type bacteriophage is a member of the family Herelleviridae such as Listeria phage LMTA-94, P100virus, and A511. Ackermannviridae, including Kuttervirus (aka (Vil-like). In some embodiments, the selected wild-type bacteriophage infects Listeria spp. In other embodiments, the selected wild-type bacteriophage is LMA4 and LMA8. The genus Pecentumvirus, under the family Herelleviridae includes bacteriophages such as Listeria phage LMSP-25, Listeria phage LMTA-148, Listeria phage LMTA-34, Listeria phage LP-048, Listeria phage LP-064, Listeria phage LP-083-2, Listeria phage LP-125, Listeria virus P100, Listeria phage List-36, Listeria phage WIL-1, Listeria phage vB_LmoM_AG20, and Listeria virus A511. LMA4 and LMA8 are also likely in the genus Pecentumvirus, under the family Herelleviridae. The family Siphoviridae includes Listeria phages A006, A118, A500, B025, LP-026, LP-030-2, LP-030-3, LP-037, LP-101, LP-110, LP-114, P35, P40, P70, PSA, vB_LmoS_188, and vB_Lmos_293.

S. aureus phages include, but are not limited to phage K, SA1, SA2, SA3, SA11, SA77, SA 187, Twort, NCTC9857, Ph5, Ph9, Ph10, Ph12, Ph13, U4, U14, U16, and U46. Well-studied phages of E. coli include T1, T2, T3, T4, T5, T7, and lambda; other E. coli phages available in the ATCC collection, for example, include phiX174, S13, Ox6, MS2, phiV1, PR772, and ZIK1. Pseudomonas aeruginosa phages may include ATCC phages Pa2, phiKZ, PB1 or phages closely related. Alternatively, natural phage may be isolated from a variety of environmental sources. A source for phage isolation may be selected based on the location where a microorganism of interest is expected to be found.

In some embodiments, the phage is derived from T7, T4, T4-like, phage K, MP131, MP115, MP112, MP506, MP87, Rambo, SAP-JV1, SAP-BZ2, PAP-WH2, PAP-WH3, PAP-JP1, PAP-JP2 or another naturally occurring phage having a genome with at least 99, 98, 97, 96, 95, 94, 93, 92, 91 90, 89, 88, 87, 86, 85, 84, 83, 82, 81, 80, 79, 78, 77, 76, 75, 74, 73, 72, 71, or 70% homology to phages disclosed above. In some aspects, the invention comprises a recombinant phage comprising an indicator gene inserted into a late gene region of the phage. In some embodiments, the phage is in the genus Tequatrovirus or Kayvirus. In one embodiment, the recombinant phage is derived from phage K, SAP-JV 1, SAP-BZ2, or MP115. In certain embodiments, the recombinant phage is highly specific for a particular bacterium. For example, in certain embodiments, the recombinant phage is highly specific for Staphylococcus aureus. In an embodiment, the recombinant phage can distinguish Staphylococcus aureus from at least 100 other types of bacteria.

In some embodiments, a sample is incubated with an indicator cocktail composition comprising at least one bacteriophage. In certain instances, the indicator cocktail composition comprises at least one bacteriophage specific for a bacteria of interest. In some embodiments, the indicator cocktail composition comprises two or more species of recombinant bacteriophages specific for the same bacteria of interest. In some embodiments, the bacteria of interest may be a genus, species, strain, or type of bacteria. In some instances, an indicator cocktail composition may be specific for a particular genus (e.g., Listeria species), a particular species (e.g., Listeria monocytogenes), strain (E. coli 0157:H7) or type (e.g., gram-positive) of bacteria. For example, an indicator cocktail composition may comprise two or more recombinant bacteriophages specific for Listeria monocytogenes. In some instances, the indicator cocktail composition comprises at least 1, 2, 3, 4, 5, 6, 7, 8, 9, or 10 different recombinant bacteriophages specific for the bacteria of interest.

In additional embodiments, a multiplex cocktail composition comprises at least two indicator cocktail compositions, wherein a first indicator cocktail composition is specific for a first bacterium of interest and a second indicator cocktail composition is specific for a second bacterium of interest. In certain embodiments, the multiplex cocktail composition comprises at least 2, 3, 4, 5, 6, 7, 8, 9, or 10 indicator cocktail compositions. For example, to determine whether any ESKAPE pathogens are present in a sample, a multiplex cocktail composition comprising a first indicator cocktail specific for Enterococcus faecium, a second indicator cocktail specific for Staphylococcus aureus, a third indicator cocktail specific for Klebsiella pneumoniae, a fourth indicator cocktail specific for Acinetobacter baumannii, a fifth indicator cocktail specific for Pseudomonas aeruginosa and a sixth indicator cocktail specific for Enterobacter species may be used.

In some embodiments, a genetic construct comprising an indicator gene encoding an indicator protein product is inserted into the bacteriophage genome. The genetic modifications may avoid deletions of wild-type genes and thus the modified phage may remain more similar to the wild-type infectious agent than many commercially available phage. Environmentally derived bacteriophage may be more specific for bacteria that are found in the environment and as such, genetically distinct from phage available commercially.

An indicator gene may express a variety of biomolecules. The indicator gene is a gene that expresses a detectable product or an enzyme that produces a detectable product. For example, in one embodiment the indicator gene encodes an indicator protein product such as a luciferase enzyme. Various types of luciferase may be used. In alternate embodiments, and as described in more detail herein, the luciferase is one of Oplophorus luciferase, Firefly luciferase, Lucia luciferase, Renilla luciferase, or an engineered luciferase. In some embodiments, the luciferase gene is derived from Oplophorus. In some embodiments, the indicator gene is a genetically modified luciferase gene, such as NANOLUC®.

Moreover, phage genes thought to be nonessential may have unrecognized function. For example, an apparently nonessential gene may have an important function in elevating burst size such as subtle cutting, fitting, or trimming functions in assembly. Therefore, deleting genes to insert an indicator gene may be detrimental. Most phages can package DNA that is a few percent larger than their natural genome. With this consideration, a smaller indicator gene may be a more appropriate choice for modifying a bacteriophage, especially one with a smaller genome. OpLuc and NANOLUC® proteins are only about 20 kDa (approximately 500-600 bp to encode), while FLuc is about 62 kDa (approximately 1,700 bp to encode). Moreover, the indicator gene should not be expressed endogenously by the bacteria (i.e., is not part of the bacterial genome), should generate a high signal to background ratio, and should be readily detectable in a timely manner. Promega's NANOLUC® is a modified Oplophorus gracilirostris (deep sea shrimp) luciferase. In some embodiments, NANOLUC® combined with Promega's NANO-GLO®, an imidazopyrazinone substrate (furimazine), can provide a robust signal with low background.

Genetic modifications to infectious agents may include insertions, deletions, or substitutions of a small fragment of nucleic acid, a substantial part of a gene, or an entire gene. In some embodiments, inserted or substituted nucleic acids comprise non-native sequences. In some embodiments, the indicator gene encodes a fusion protein. A non-native indicator gene may be inserted into a bacteriophage genome such that it is under the control of an exogenous bacteriophage promoter. Thus, in some embodiments, the non-native indicator gene is not part of a fusion protein. That is, in some embodiments, a genetic modification may be configured such that the indicator protein product does not comprise polypeptides of the wild-type bacteriophage. In some embodiments, the indicator protein product is soluble.

In some embodiments, the indicator gene encodes an indicator protein that is capable of being captured or immobilized. In some instances the indicator gene encodes an indicator protein that is specific to a binding partner. Thus, in certain embodiments, the indicator protein product is capable of being captured using an immobilized binding partner. The binding partner may be any molecule specific for and capable of binding the indicator protein. Suitable binding partners for use with the compositions, methods, systems, and kits described herein, include but are not limited to peptides, antibodies, aptamers, ligands, proteins, or any portion thereof. In certain embodiments, the immobilized binding partner is a capture antibody. In some embodiments, the indicator protein product is capable of being captured using an immobilized antibody or fragment thereof specific to the indicator protein product. For example, the indicator protein may be a luciferase and the luciferase may be specific to an anti-luciferase antibody.

In other embodiments, the genetic construct comprises genes for an indicator protein and a capture moiety, wherein the indicator protein and capture moiety genes encode a fusion protein. In certain instances, the indicator gene and capture moiety encode an indicator protein-capture moiety fusion protein. In some instances, the capture moiety is capable of being captured using an immobilized binding partner. The binding partner may be any molecule specific for and capable of binding the indicator protein. Suitable binding partners for use with the compositions, methods, systems, and kits described herein, include but are not limited to antibodies, aptamers, ligands, or any portion thereof. In certain embodiments, the immobilized binding partner is a capture antibody. Thus, in some embodiments, the capture moiety is capable of being captured using an immobilized antibody or fragment thereof specific to the capture moiety.

In certain embodiments, the capture moiety is a peptide tag. The peptide tag may comprise less than 50 amino acid residues, for example, less than 50, 40, 30, 20, 10, or 5 amino acid residues. In certain embodiments, the peptide tag may comprise 20, 19, 18, 17, 16, 15, 14, 13, 12, 11, 10, 9, 8, 7, 6, 5, 4, 3, or 2 amino acid residues. The relatively small size of the peptide tag is advantageous in that it may prevent the peptide tag from interfering with the function or activity of the labeled protein. Thus, the peptide-tagged indicator protein product will maintain its ability to fold and function. In some embodiments, the capture moiety (e.g., peptide tag) exhibits high binding activity and specificity to an immobilized binding partner. The capture moiety is specific for the binding partner and avoids binding to a significant percentage of non-target binding partners. In some embodiments, a capture moiety avoids binding greater than about 90%, 91%, 92%, 93%, 94%, 95%, 96%, 97%, 98%, 99% or greater of non-target binding partners. There several peptide tag systems known in the art and that are commercially available, including but not limited to, glutathione S-transferase (GST), c-myc, FLAG, HA, six histidine (His6), T7-Tag, Strep-Tag, and Avi-Tag.

In some embodiments of the recombinant indicator phage, the genetic construct further comprises an exogenous promoter. The additional, exogenous late promoter (class III promoter, e.g., from phage K or T7 or T4) has high affinity for RNA polymerase of the same native phage (e.g., phage K or T7 or T4, respectively) that transcribes genes for structural proteins assembled into the phage particle. These proteins are the most abundant proteins made by the phage, as each phage particle comprises dozens or hundreds of copies of these molecules. The use of a viral late promoter can ensure optimally high level of expression of the indicator protein product. The use of a late viral promoter derived from, specific to, or active under the original wild-type phage the indicator phage is derived from (e.g., the phage K or T4 or T7 late promoter with a phage K- or T4- or T7-based system) can further ensure optimal expression of the enzyme. The use of a standard bacterial (non-viral/non-phage) promoter may in some cases be detrimental to expression, as these promoters are often down-regulated during phage infection (in order for the phage to prioritize the bacterial resources for phage protein production). Thus, in some embodiments, the phage is engineered to encode and express at high levels an indicator protein product.

In some embodiments, expression of the indicator gene in progeny bacteriophage following infection of host bacteria results in a free, soluble protein product. In some embodiments, the non-native indicator gene is not contiguous with a gene encoding a structural phage protein and therefore does not yield a fusion protein. Unlike systems that employ a fusion of an indicator protein product to a phage structural protein (i.e., a fusion protein), some embodiments of the present invention express a soluble or free indicator (e.g., soluble or free luciferase). In some embodiments, the indicator protein is free of the bacteriophage structure. That is, the indicator protein is not attached or tethered to a phage structural protein. As such, the gene for the indicator is not fused with other genes in the recombinant phage genome. This may greatly increase the sensitivity of the assay (down to a single bacterium), and simplify the assay, allowing the assay to be completed in two hours or less for some embodiments, as opposed to several hours due to additional purification steps required with constructs that produce detectable fusion proteins. To clarify, the fusion of an indicator with a peptide or protein tag, where the indicator-peptide or indicator-protein fusion produced is free of structural attachments (i.e., untethered) to the bacteriophage, is a free or soluble indicator.

In other embodiments, expression of the indicator gene in progeny bacteriophage following infection of host bacteria results in an indicator protein-capture moiety fusion protein product. In some embodiments, the fusion indicator protein is not fused to any bacteriophage structural proteins. Indicator protein-bacteriophage structural protein fusion proteins (e.g., luciferase-capsid protein fusion protein) by definition limit the number of the moieties attached to subunits of a protein in the bacteriophage. For example, using a commercially available system designed to serve as a platform for a fusion protein would result in about 415 copies of the fusion moiety, corresponding to the about 415 copies of the gene 10B capsid protein in each T7 bacteriophage particle. Without this constraint, infected bacteria can be expected to express many more copies of the indicator protein product (e.g., luciferase) than can fit on the bacteriophage. Additionally, large fusion proteins, such as a capsid-luciferase fusion, may inhibit assembly of the bacteriophage particle, thus yielding fewer bacteriophage progeny. Thus, in some embodiments, the fusion indicator protein-capture moiety fusion product is free of bacteriophage structural proteins.

In some embodiments, the indicator phage encodes an indicator protein, such as a detectable enzyme. The indicator gene product may generate light and/or may be detectable by a color change. Various appropriate enzymes are commercially available, such as alkaline phosphatase (AP), horseradish peroxidase (HRP), or luciferase (Luc). In some embodiments, these enzymes may serve as the indicator protein product. In some embodiments, Firefly luciferase is the indicator protein product. In some embodiments, Oplophorus luciferase is the indicator moiety. In some embodiments, NANOLUC® is the indicator protein product. Other engineered luciferases or other enzymes that generate detectable signals may also be appropriate indicator moieties.

In some embodiments, the use of non-indicator protein-bacteriophage structural protein fusion protein product eliminates the need to remove contaminating stock phage from the lysate of the infected sample cells. With a structural bacteriophage fusion protein system, any bacteriophage used to infect sample cells would have the indicator protein product attached, and would be indistinguishable from the daughter bacteriophage also containing the indicator protein product. As detection of sample bacteria relies on the detection of a newly created (de novo synthesized) indicator protein product, using structural bacteriophage fusion protein constructs requires additional steps to separate old (stock phage) indicator from newly synthesized indicator. This may be accomplished by washing the infected cells multiple times, prior to the completion of the bacteriophage life cycle, inactivating excess stock phage after infection by physical or chemical means, and/or chemically modifying the stock bacteriophage with a binding moiety (such as biotin), which can then be bound and separated (such as by Streptavidin-coated Sepharose beads). However, even with all these attempts at removal, stock phage can remain when a high concentration of stock phage is used to assure infection of a low number of sample cells, creating background signal that may obscure detection of signal from infected cell progeny phage.

By contrast, with the soluble, non-structural bacteriophage fusion protein products expressed in some embodiments of the present invention, purification of the stock phage from the final lysate is unnecessary, as the stock phage compositions do not have any indicator protein product. Thus, any indicator protein product present after infection must have been created de novo, indicating the presence of an infected bacterium or bacteria. To take advantage of this benefit, the production and preparation of phage may include purification of the phage from any free indicator protein product produced during the production of recombinant bacteriophage in bacterial culture. Standard bacteriophage purification techniques may be employed to purify some embodiments of phage according to the present invention, such as sucrose density gradient centrifugation, cesium chloride isopycnic density gradient centrifugation, HPLC, size exclusion chromatography, and dialysis or derived technologies (such as Amicon brand concentrators—Millipore, Inc.). Cesium chloride isopycnic ultracentrifugation can be employed as part of the preparation of recombinant phage of the disclosure, to separate stock phage particles from contaminating luciferase protein produced upon propagation of the phage in the bacterial host. In this way, the recombinant bacteriophages of the invention are substantially free of any luciferase generated during production in the bacteria. Removal of residual luciferase present in the phage stock can substantially reduce background signal observed when the recombinant bacteriophages are incubated with a test sample.

In some embodiments of the modified recombinant bacteriophage, the late promoter (class III promoter) has high affinity for RNA polymerase of the same bacteriophage that transcribes genes for structural proteins assembled into the bacteriophage particle. These proteins are the most abundant proteins made by the phage, as each bacteriophage particle comprises dozens or hundreds of copies of these molecules. The use of a viral late promoter can ensure optimally high level of expression of the luciferase indicator protein product. The use of a late viral promoter derived from, specific to, or active under the original wild-type bacteriophage the indicator phage is derived from can further ensure optimal expression of the indicator protein product. For example, indicator phage specific for MRSA may comprise the consensus late gene promoter from S. aureus phage ISP. In other instances the SAP-BZ2 may comprise a Gram positive/SigA promoter consensus region. The use of a standard bacterial (non-viral/non-bacteriophage) promoter may in some cases be detrimental to expression, as these promoters are often down-regulated during bacteriophage infection (in order for the bacteriophage to prioritize the bacterial resources for phage protein production). Thus, in some embodiments, the phage is preferably engineered to encode and express at high level a soluble (free) indicator protein or soluble (free) capture moiety-indicator protein fusion product, using a placement in the genome that does not limit expression to the number of subunits of a phage structural component.

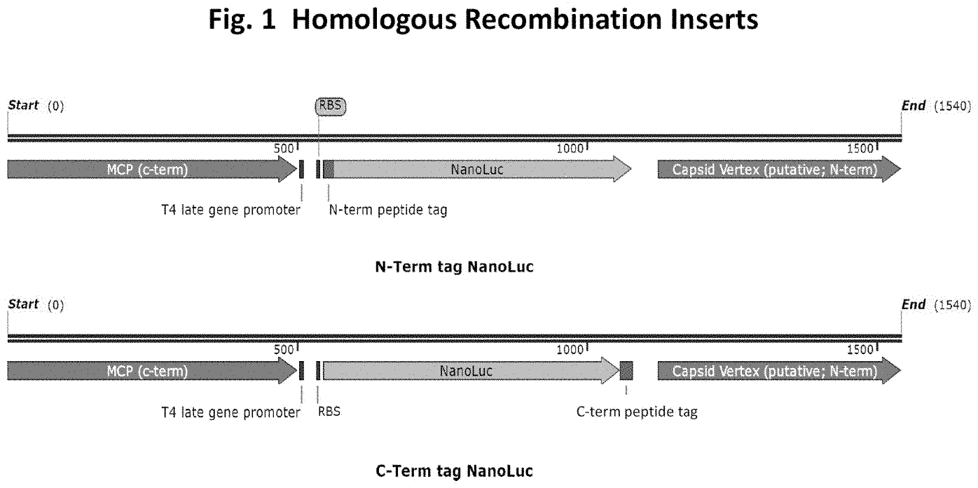

As shown in FIG. 1, a genetic construct may include a homologous recombination insert comprising an indicator gene such as a luciferase gene. The genetic construct can include a peptide tag placed on the N-terminal or C-terminal end of the indicator gene. In some embodiments, the construct may also include a bacteriophage late gene promoter and a ribosomal binding site (RBS). The promoter depicted is in addition to and separate from the endogenous late gene promoter upstream of the endogenous late genes, such as the gene for major capsid protein (MCP).

Compositions of the disclosure may comprise one or more wild-type or genetically modified infectious agents (e.g., bacteriophages) and one or more indicator genes. In some embodiments, compositions can include cocktails of different indicator phages that may encode and express the same or different indicator proteins. In some embodiments, the cocktail of indicator bacteriophages comprises at least two different types of recombinant bacteriophages.

In some indicator phage embodiments, the genetic construct is inserted into an untranslated region to avoid disruption of functional genes, leaving wild-type phage genes intact, which may lead to greater fitness when infecting non-laboratory strains of bacteria. Additionally, including stop codons in all three reading frames may help to increase expression by reducing read-through, also known as leaky expression. This strategy may also eliminate the possibility of a structural bacteriophage fusion protein being made at low levels, which would manifest as background signal (e.g., luciferase) that cannot be separated from the phage.

Thus, in some embodiments, the present invention comprises a genetically modified bacteriophage comprising a genetic construct comprising a non-bacteriophage indicator gene in the late (class III) gene region. In some embodiments, the genetic construct further comprises a capture moiety that is specific for a binding partner. In some instances the genetic construct further comprises an exogenous, late promoter such that the indicator gene and the capture moiety are under the control of the late promoter. Using a viral late gene promoter ensures the indicator gene (e.g., luciferase) is not only expressed at high levels, like viral capsid proteins, but also does not shut down like endogenous bacterial genes or even early viral genes.

Some embodiments include designing (and optionally preparing) a sequence for homologous recombination downstream of the major capsid protein gene. Other embodiments include designing (and optionally preparing) a sequence for homologous recombination upstream of the major capsid protein gene. In some embodiments, the sequence comprises a codon-optimized indicator gene preceded by an untranslated region. The untranslated region may include a phage late gene promoter and ribosomal entry site.

Methods of Using Bacteriophages for the Detection of Bacteria

As noted herein, in certain embodiments, the invention may comprise methods of using modified infectious agents for detecting microorganisms. The methods of the disclosure may be embodied in a variety of ways.

In one aspect, a method of detecting a bacteria of interest in a sample comprises the steps of: (i) obtaining the sample; (ii) incubating the sample with at least one indicator cocktail composition, wherein the recombinant bacteriophage comprises a genetic construct inserted into the bacteriophage genome, and wherein the genetic construct comprises an indicator gene encoding an indicator protein product; (iii) capturing the indicator protein product; and (iv) detecting the captured indicator protein product, wherein detection of the captured indicator protein product indicates the bacteria of interest is present in the sample.

In another aspect, a method of detecting a bacteria of interest in a sample comprising the steps of: (i) obtaining the sample; (ii) incubating the sample with at least one indicator cocktail composition, wherein the recombinant bacteriophage comprises a genetic construct inserted into the bacteriophage genome, and wherein the genetic construct comprises an indicator gene and a capture moiety encoding a capture moiety-indicator protein fusion product; (iii) capturing the capture moiety-indicator protein fusion product; and (iv) detecting the captured indicator protein product, wherein detection of the captured indicator protein product indicates the bacteria of interest is present in the sample.

In certain embodiments, the method for detecting a bacteria of interest comprises detecting at least one bacteria of interest. In an embodiment, the method for detecting at least one bacteria of interest in a sample comprises the step of obtaining the sample. The sample may be medical, veterinary, pharmaceutical, clinical, laboratory, environmental, food, or water or any combination thereof as described in detail above.

In some embodiments, enrichment of bacteria in the sample is not needed prior to testing. In some embodiments, the sample may be enriched prior to testing by incubation in conditions that encourage growth. In such embodiments, the enrichment period can be 1, 2, 3, 4, 5, 6, 7, 8, 9, 10, 11, 12, 13, 14, 15, 16, 17, 18, 19, 20, 21, 22, 23, or 24 hours or longer, depending on the sample type and size.

In some embodiments, the sample is a biological sample, including but not limited to urine, nasal, mucus, saliva, blood, sputum, cerebrospinal fluid, and fecal samples and different types of swabs. In certain embodiments, the blood sample may be whole blood, serum, or plasma. In some instances, the biological sample requires no additional processing steps prior to detection of bacteria. In some embodiments, the biological sample is further enriched prior to detection of the bacteria. In some instances the sample is enriched for a relatively short period of time. For example, whole blood samples may be enriched for less than 8, 7, 6, 5, 4, 3, 2, 1, or 0.5 hours. In some embodiments, the whole blood is diluted in media. For example, a whole blood sample may be diluted to 25:75 (whole blood:media).

In certain embodiments, the method for detecting at least one bacteria of interest in a sample comprises the step of incubating the sample with an indicator cocktail composition comprising at least one recombinant bacteriophage. In certain instances, the indicator cocktail composition comprises at least one bacteriophage specific for a bacteria of interest. In some embodiments, the indicator cocktail composition comprises two or more species of recombinant bacteriophages specific for the same bacteria of interest. In some embodiments, the bacteria of interest may be a genus, species, strain, or type of bacteria as described in detail herein. In some instances, the indicator cocktail composition comprises at least 1, 2, 3, 4, 5, 6, 7, 8, 9, or 10 different recombinant bacteriophages specific for the bacteria of interest.

In additional embodiments, a multiplex cocktail composition comprises at least two indicator cocktail compositions, wherein a first indicator cocktail composition is specific for a first bacteria of interest and a second indicator cocktail composition is specific for a second bacteria of interest. In certain embodiments, the multiplex cocktail composition comprises at least 2, 3, 4, 5, 6, 7, 8, 9, or 10 indicator cocktail compositions as described in detail herein.

The bacteriophage may be engineered to express a soluble (non-structural bacteriophage fusion) luciferase during replication of the phage. Expression of luciferase is driven by a viral capsid promoter (e.g., the bacteriophage Pecentumvirus or T4 late promoter), yielding high expression. Stock phage are prepared such that they are free of luciferase, so the luciferase detected in the assay must come from replication of progeny phage during infection of the bacterial cells. Thus, there is generally no need to separate out the parental phage from the progeny phage.

In some embodiments, the recombinant bacteriophage comprises a genetic construct comprising an indicator gene encoding an indicator protein product. An indicator gene may express a variety of biomolecules. For example, in one embodiment the indicator gene encodes a luciferase enzyme. Various types of luciferase may be used as described in detail herein.

In some embodiments, the genetic construct further comprises a capture moiety. In other embodiments, the genetic construct comprises genes for an indicator protein and a capture moiety, wherein the indicator protein and capture moiety encode a fusion protein. In some instances, the capture moiety is capable of being captured using an immobilized binding partner. The binding partner may be any molecule specific for and capable of binding the indicator protein. Suitable binding partners for use with the compositions, methods, systems, and kits described herein, include but are not limited to peptides, antibodies, aptamers, ligands, or any portion thereof. In certain embodiments, the immobilized binding partner is a capture antibody. Thus, in some embodiments, the capture moiety is capable of being captured using an immobilized antibody or fragment thereof specific to the capture moiety.

In some embodiments, the immobilized binding partner (e.g., an antibody) is covalently coupled to a surface. In certain instances, the surface comprises amino or carboxyl groups. Amino or carboxyl groups can be used to covalently couple the immobilized binding partner to the surface.

In certain embodiments, the capture moiety is a peptide tag, The peptide tag may comprise less than 500 amino acid residues, for example, less than 500, 450, 400, 350, 300, 250, 200, 100, 50, 40, 30, 20, 10, or 5 amino acid residues. In certain embodiments, the peptide tag may comprise 10, 9, 8, 7, 6, 5, 4, 3, or 2 amino acid residues. The relatively small size of the peptide tag is advantageous in that it may prevent the peptide tag from interfering with the function or activity of the labeled protein. Thus, the peptide-tagged indicator protein product will maintain its ability to fold and function.

In some embodiments, the genetic construct further comprises an additional exogenous promoter. The additional, exogenous late promoter (class III promoter, e.g., from phage K or T7 or T4) has high affinity for RNA polymerase of the same native phage (e.g., phage K or T7 or T4, respectively) that transcribes genes for structural proteins assembled into the phage particle as described in detail herein.

Thus, in some embodiments, the method for detecting at least one bacteria of interest in a sample comprises the step of incubating the sample with an indicator cocktail composition comprising at least one recombinant bacteriophage, wherein the recombinant bacteriophage comprises a genetic construct comprising: (i) an indicator gene encoding an indicator protein product; (ii) a capture moiety; and (iii) an exogenous promoter.

In some embodiments, the sample is incubated with the indicator cocktail composition for at least 0.5, 1, 2, 3, 4, 5, 6, 7, 8, 9, or 10 hours. In some embodiments, the sample is incubated with the multiplex cocktail compositions for at least 0.5, 1, 2, 3, 4, 5, 6, 7, 8, 9, or 10 hours. If the bacteria of interest are present in the sample, the recombinant bacteriophages will infect the bacteria, thereby expressing the indicator gene. Thus, in some embodiments, the method comprises treatment with lysis buffer to lyse the bacteria infected with the recombinant bacteriophages prior to capturing the indicator protein product or the capture moiety-indicator protein product fusion.

In some embodiments, the surface may comprise an immobilized binding partner. For example, one or more specific recognition elements can be immobilized in discrete areas of a surface in order to generate an array for analyte recognition. The indicator protein product can be brought into contact with the surface comprising the immobilized binding partner. In some embodiments, several different binding partners can be immobilized simultaneously on one surface. In some embodiments, the immobilized binding partner is an antibody or a fragment thereof.

In some embodiments, one or more different immobilized binding partners can be deposited (e.g., pipetted) on a surface (e.g., a plate) for capturing the indicator protein product. In some aspects, the surface can improve accessibility and capture of the indicator protein product by orienting immobilized binding partners. For example, an antibody can be deposited on a plate and incubated for a period of time. In some embodiments, the antibody can be rabbit or antibodies goat antibodies. Optionally, the plate can be washed after incubation. Subsequently, a NANOLUC® antibody can deposited on the coated plated. In some aspects, it is advantageous if the amount of the indicator protein product to be deposited on a surface with an immobilized binding partner is equal to or less than the amount of immobilized binding partner for the formation of a monolayer on the surface as a solid support. For example, the immobilized binding partner can be antibodies that are bound to a layer on the surface of a solid support, resulting in accessibility of their specific binding epitopes.

In some embodiments, the method includes adding a protein to the antibodies to promote infection by bacteriophage. Bacteria (e.g., S. aureus) bind antibodies (e.g., IgG) in the blood preventing bacteriophage from infecting the cells. In some embodiments, Protein A is added to bind the antibodies in the blood thereby preventing the antibodies from binding to bacteria. When bacteria cells divide in the presence of Protein A, the antibodies cannot bind to the daughter cells, allowing infection of the cells in the blood by the bacteriophage. In some embodiments, Protein A is added to a phage cocktail. For example, Protein A can be mixed with the phage cocktail prior to infection.

In some embodiments, the methods of the disclosure may comprise various other steps to increase sensitivity. The sensitivity of the method of detecting a bacterium of interest may be increased by one or more washing steps. For example, the method may comprise a step for washing the captured indicator protein product to remove excess bacteriophage and/or luciferase or other indicator protein contaminating the bacteriophage preparation. Additionally, captured microorganisms may be washed following incubation with antibiotic and the infectious agent, prior to addition of lysis buffer and substrate. These additional washing steps aid in the removal of excess parental phage and/or luciferase or other indicator protein contaminating the phage preparation. In some embodiments, a microorganism can be captured, washed, and then infected with the bacteriophage.

In some embodiments, the method for detecting at least one bacteria of interest in a sample comprises the step of capturing the indicator protein product. In certain embodiments, capturing the indicator protein product comprises contacting the sample with a surface. For example, the surface may be a microtiter plate, latex particle, lateral flow strip, bead, magnetic particle, dipstick, microsphere or any other surface capable of capturing the indicator protein product. In certain instances, the indicator protein product may adhere or bind to the surface during the capture step.

In some embodiments the surface comprises a capture area comprising an immobilized binding partner. In some embodiments, the binding partner is specific for the indicator protein product. In other embodiments, the binding partner is specific for the capture moiety fused to the indicator protein.

In some embodiments, the binding partner is directly immobilized on the surface. For example, the capture area may comprise a binding partner, wherein the binding partner is an antibody specific for the capture moiety. In other embodiments, the binding partner is indirectly immobilized on the surface. For example, the capture area may comprise a primary antibody that is specific for a binding partner that is a secondary antibody that is specific for the capture moiety. Indirectly immobilizing the binding partner on the surface may be advantageous in that it allows the binding partner to be oriented in such a way that promotes binding and capture of the capture moiety, thereby increasing the sensitivity of the assay.

The binding partner may be any molecule specific for and capable of capturing the indicator protein or the capture moiety-indicator protein fusion. For example, in some embodiments, the surface may be coated with Protein A. In other embodiments, the surface may be coated with streptavidin or avidin.

The binding partner is specific for the capture moiety and avoids binding to a significant percentage of non-target capture moieties. In some embodiments, a binding partner avoids binding greater than about 90%, 91%, 92%, 93%, 94%, 95%, 96%, 97%, 98%, 99% or greater of non-target capture moieties.

In some embodiments, the immobilized binding partner may be a protein, including but not limited to antibodies, ligands, and nucleic acids specific to the capture moiety. For example, in some embodiments, the immobilization binding partner may be an antibody specific for a peptide tag.

In some embodiments the binding partner is an antibody or a fragment thereof. An antibody is an immunoglobulin that binds specifically to a particular antigen. Antibodies used with the methods, systems, and kits described herein may be naturally or synthetically produced or engineered. In some embodiments, the antibody may be monoclonal or polyclonal. An antibody may be a member of any immunoglobulin class, including any of the human classes: IgG, IgM, IgA, and IgD. In some embodiments, an antibody is produced by chemical synthesis. In some embodiments, an antibody is derived from a mammal. In some embodiments, an antibody is derived from an animal such as, but not limited to, mouse, rat, horse, pig, rabbit, or goat. In some embodiments, an antibody is produced using a recombinant cell culture system. In some embodiments, an antibody may be a purified antibody (for example, by immune-affinity chromatography). In some embodiments, the antibody is biotinylated.

In some embodiments, the surface comprises more than one capture area, thereby allowing for multiplex detection of bacteria. In some embodiments, the surface comprises at least 2, 3, 4, 5, 6, 7, 8, 9, 10, 15, 20, 25, 50, 75, or 100 capture areas. In certain instances, each of the one or more capture areas comprises a different immobilization binding partner. For example, if the surface is a lateral flow strip, each test line (i.e., capture area) comprises a different immobilized binding partner.

In some embodiments, the surface may further comprise a control area. The control area is used to ensure assay reagents are working. The control area may be a positive control area or a negative control area.

In some embodiments, the method for detecting at least one bacteria of interest in a sample comprises the step of detecting the captured indicator protein product. In some instances, the step of detecting the captured indicator protein product comprises detecting a change in color or bioluminescence. In some embodiments, the indicator protein product encoded by the recombinant indicator bacteriophage may be detectable during or after replication of the bacteriophage. Many different types of detectable biomolecules suitable for use as indicator proteins are known in the art, and many are commercially available. In some embodiments the indicator phage comprises an indicator gene encoding an enzyme, which serves as the indicator protein. In some embodiments, the genome of the indicator phage is modified to encode a soluble protein. In other embodiments, the genome of the indicator phage is modified to encode an indicator protein-capture moiety fusion protein. In some embodiments, the indicator phage encodes a detectable enzyme. Various appropriate enzymes are commercially available, such as alkaline phosphatase (AP), horseradish peroxidase (HRP), or luciferase (Luc). In some embodiments, these enzymes may serve as the indicator protein. In some embodiments, Firefly luciferase is the indicator protein. In some embodiments, Oplophorus luciferase is the indicator protein. In some embodiments, NANOLUC® is the indicator protein. Other engineered luciferases or other enzymes that generate detectable signals may also be appropriate indicator proteins.

In some embodiments, the indicator gene encodes a protein that emits an intrinsic signal, such as a fluorescent protein (e.g., green fluorescent protein or others). The indicator may emit light and/or may be detectable by a color change. In some embodiments, the indicator gene encodes an enzyme (e.g., luciferase) that interacts with a substrate to generate signal. In some embodiments, the indicator gene is a luciferase gene. In some embodiments, the luciferase gene is one of Oplophorus luciferase, Firefly luciferase, Renilla luciferase, External Gaussia luciferase, Lucia luciferase, or an engineered luciferase such as NANOLUC®, Rluc8.6-535, or Orange Nano-lantern. Thus in some instances, the step for detecting the captured indicator protein product further comprises reacting a substrate with the indicator protein. For example, NANOLUC® combined with Promega's NANO-GLO®, an imidazopyrazinone substrate (furimazine), can provide a robust signal with low background.

Detecting the captured indicator protein may include detecting emissions of light. In some embodiments, the indicator protein product (e.g., luciferase) is reacted with a substrate to produce a detectable signal. The detection of the signal can be achieved with any machine or device generally known in the art. In some embodiments, the signal can be detected using an In Vivo Imaging System (IVIS). The IVIS uses a CCD camera or a CMOS sensor to measure light emissions by total flux. Total flux=radiance (photons/second). Average radiance is measured as photons/second/cm²/steradian. In other embodiments, the detection of the signal can be achieved with a luminometer, a spectrophotometer, CCD camera, or CMOS camera may detect color changes and other light emissions. In some embodiments the signal is measured as absolute RLU. In further embodiments, the signal to background ratio needs to be high (e.g., >2.0, >2.5, or >3.0) in order for single cells or low numbers of cells to be detected reliably.

In certain instances, detection of the captured indicator protein product indicates the genus, species, strain, or type of bacteria present in the sample. Detection of a particular bacteria of interest in a sample is dependent upon the specificity of the recombinant bacteriophage or the cocktail of recombinant bacteriophages. For example, a recombinant bacteriophage indicator cocktail composition may be specific for a particular genus (e.g., Listeria species), a particular species (e.g., Listeria monocytogenes), a particular strain (E. coli 0157:H7) or a particular type (e.g., gram-positive bacteria) of bacteria. Thus, detection of an indicator protein using a recombinant bacteriophage indicator cocktail composition indicates which bacteria of interest are present in a sample. The bacteriophages of the indicator cocktail composition may be selected, in part, based on the inclusivity and exclusivity of each bacteriophage to ensure specificity for the bacteria of interest.

In some embodiments, at least one aliquot of a biological sample is contacted with an amount of an indicator bacteriophage cocktail composition. In certain instances, the indicator cocktail composition comprises at least one recombinant bacteriophage specific for a particular microorganism of interest. In other embodiments, the indicator cocktail composition comprises at least two, at least three, at least four, at least five, at least six, at least seven, at least eight, at least nine, or at least ten types of recombinant bacteriophages specific for a particular microorganism of interest. In certain embodiments, the method further comprises contacting a plurality of aliquots of the biological samples with a plurality of indicator cocktail compositions. In some instances, each indicator cocktail composition is specific for a different microorganism of interest. For example, a first aliquot may be contacted with a recombinant bacteriophage cocktail composition specific for Enterococcus faecalis, a second aliquot may be contacted with a recombinant bacteriophage cocktail composition specific for Staphylococcus aureus, a third aliquot may be contacted with a recombinant bacteriophage cocktail composition specific for Staphylococcus epidermidis, a fourth aliquot may be contacted with a recombinant bacteriophage cocktail composition specific for Streptococcus viridans, a fifth aliquot may be contacted with a recombinant bacteriophage cocktail composition specific for Escherichia coli, a sixth aliquot may be contacted with a recombinant bacteriophage cocktail composition specific for Klebsiella pneumoniae, a seventh aliquot may be contacted with a recombinant bacteriophage cocktail composition specific for Proteus mirabilis, and an eighth aliquot may be contacted with a recombinant bacteriophage cocktail composition specific for Pseudomonas aeruginosa. In some embodiments, at least 1, 2, 3, 4, 5, 6, 7, 8, 9, 10, 11, 12, 13, 14, 15, 16, 17, 18, 19, 20, 21, 22, 23, 24, or 25 aliquots of the biological sample are contacted with at least 1, 2, 3, 4, 5, 6, 7, 8, 9, 10, 11, 12, 13, 14, 15, 16, 17, 18, 19, 20, 21, 22, 23, 24, or 25 different indicator cocktail compositions. In some embodiments, the cocktail composition comprises two or more bacteriophages specific for the same microorganism of interest.

In some embodiments, the method is capable of detecting more than one bacteria of interest in a sample. In some instances, the methods described herein are used for a multiplex assay. A multiplex assay allows for simultaneous detection of multiple genus, species, strains, or types of bacteria present in a sample.

For example, to determine whether any ESKAPE pathogens are present in a sample, the sample may be incubated with a multiplex cocktail composition comprising a first indicator cocktail specific for Enterococcus faecium, a second indicator cocktail specific for Staphylococcus aureus, a third indicator cocktail specific for Klebsiella pneumoniae, a fourth indicator cocktail specific for Acinetobacter baumannii, a fifth indicator cocktail specific for Pseudomonas aeruginosa and a sixth indicator cocktail specific for Enterobacter species may be used, wherein the bacteriophages of each indicator cocktail composition comprise a unique tag (i.e., Enterococcus faecium phage cocktail has tag A, Staphylococcus aureus phage cocktail has tag B, Klebsiella pneumoniae phage cocktail has tag C, Acinetobacter baumannii phage cocktail has tag D, Pseudomonas aeruginosa phage cocktail has tag E, and Enterobacter species phage cocktail has tag F). Following incubation with the multiplex cocktail composition, the sample may be lysed and then added to a lateral flow strip, wherein the lateral flow strip comprises six capture areas, wherein each capture area comprises a binding partner specific for each of the unique tags (i.e., capture moieties). Thus, if a particular bacteria is present in the sample, the capture moiety-indicator protein fusion will be captured in the capture area specific to the capture moiety corresponding to the bacteria of interest. The indicator protein can then be detected as described in detail herein, thus, indicating which bacteria are present in the sample.

In some embodiments, the assay may be performed to utilize a general concept that can be modified to accommodate different sample types or sizes and assay formats. Embodiments employing recombinant bacteriophage of the invention (i.e., indicator bacteriophage) may allow rapid detection of specific bacterial strains with total assay times under 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12, 12.5, 13.0, 13.5, 14.0, 14.5, 15.0, 15.5, 16.0, 16.5, 17.0, 17.5, 18.0, 18.5, 19.0, 19.5, 20.0, 21.0, 21.5 22.0, 22.5, 23.0, 23.5, 24.0, 24.5 25.0, 25.5, or 26.0 hours, depending on the sample type, sample size, and assay format. For example, the amount of time required may be somewhat shorter or longer depending on the strain of bacteriophage and the strain of bacteria to be detected in the assay, type and size of the sample to be tested, conditions required for viability of the target, complexity of the physical/chemical environment, and the concentration of “endogenous” non-target bacterial contaminants. For example, detection for the presence of Gram-negative strains (e.g., E. coli, Klebsiella, Shigella) may be completed with total assay times under 0.5, 1.0, 1.5, 2.0, 2.5, or 3.0 hours without detecting for antibiotic resistance or total assay times under 1.5, 2.0, 2.5, 3.0, 3.5, 4.0, or 4.5 hours with detecting for antibiotic resistance. Detection for the presence of Gram-positive strains may be completed with total assay times under 1.0, 1.5, 2.0, 2.5, 3.0, 3.5, 4.0, or 4.5 hours without detecting antibiotic resistance or 4.0, 4.5, 5.0, 5.5, 6.0, 6.5 7, 7.5, or 8 hours with detecting antibiotic resistance.

In some embodiments, the indicator bacteriophage comprises a detectable indicator protein product, and infection of a single pathogenic cell (e.g., bacterium) can be detected by an amplified signal generated via the indicator protein product. Thus, the method may comprise detecting an indicator protein product produced during phage replication, wherein detection of the indicator indicates that the bacterium of interest is present in the sample.

In an embodiment, the invention may comprise a method for detecting a bacterium of interest in a sample comprising the steps of: incubating the sample with a recombinant bacteriophage that infects the bacterium of interest, wherein the recombinant bacteriophage comprises an indicator gene inserted into a late gene region of the bacteriophage such that expression of the indicator gene during bacteriophage replication following infection of host bacteria results in production of a soluble indicator protein product; and detecting the indicator protein product, wherein positive detection of the indicator protein product indicates that the bacterium of interest is present in the sample. In some embodiments, the amount of indicator protein product detected corresponds to the amount of the bacterium of interest present in the sample. In some embodiments, the indicator phage detection assay is able to detect and quantify the number of viable microorganisms present in the sample.

As described in more detail herein, the compositions, methods, kits and systems of the disclosure may utilize a range of concentrations of parental indicator bacteriophage to infect bacteria present in the sample. In some embodiments the indicator bacteriophage are added to the sample at a concentration sufficient to rapidly find, bind, and infect target bacteria that are present in very low numbers in the sample, such as ten cells. In some embodiments, the phage concentration can be sufficient to find, bind, and infect the target bacteria in less than one hour.

In other embodiments, these events can occur in less than two hours, or less than three hours, or less than four hours, following addition of indicator phage to the sample. For example, in certain embodiments, the bacteriophage concentration for the incubating step is greater than 1×10² PFU/mL, greater than 1×10³ PFU/mL, greater than 1×10⁴ PFU/mL greater than 1×10⁵ PFU/mL, greater than 1×10⁶ PFU/mL, or greater than 1×10⁷ PFU/mL, or greater than 1×10⁸ PFU/mL.

In certain embodiments, the recombinant stock phage composition may be purified so as to be free of any residual indicator protein that may be generated upon production of the phage stock. Thus, in certain embodiments, the recombinant bacteriophage may be purified using a sucrose gradient or cesium chloride isopycnic density gradient centrifugation prior to incubation with the sample. When the infectious agent is a bacteriophage, this purification may have the added benefit of removing bacteriophages that do not have DNA (i.e., empty phage or “ghosts”).

In some embodiments of the methods of the invention, the microorganism may be detected without any isolation or purification of the microorganisms from a sample. For example, in certain embodiments, a sample containing one or a few microorganisms of interest may be applied directly to an assay container such as a spin column, a tube, a microtiter well, or a filter and the assay is conducted in that assay container. Various embodiments of such assays are disclosed herein.

Aliquots of a test sample may be distributed directly into wells of a multi-well plate, indicator phage may be added, and after a period of time sufficient for infection, a lysis buffer may be added as well as a substrate for the indicator moiety (e.g., luciferase substrate for a luciferase indicator) and assayed for detection of the indicator signal. Some embodiments of the method can be performed on filter plates or 96 well plates. Some embodiments of the method can be performed with or without concentration of the sample before infection with indicator phage.

For example, in some embodiments, multi-well plates are used to conduct the assays. The choice of plates (or any other container in which detecting may be performed) may affect the detecting step. For example, some plates may include a colored or white background, which may affect the detection of light emissions. Generally speaking, white plates have higher sensitivity but also yield a higher background signal. Other colors of plates may generate lower background signal but also have a slightly lower sensitivity. Additionally, one reason for background signal is the leakage of light from one well to another, adjacent well. There are some plates that have white wells but the rest of the plate is black. This allows for a high signal inside the well but prevents well-to-well light leakage and thus may decrease background. Thus the choice of plate or other assay vessel may influence the sensitivity and background signal for the assay.

Methods of the disclosure may comprise various other steps to increase sensitivity. For example, as discussed in more detail herein, the method may comprise a step for washing the infected bacterium, after adding the bacteriophage but before incubating, to remove excess bacteriophage and/or luciferase or other indicator protein contaminating the bacteriophage preparation. In some embodiments, samples may be washed after incubation with the recombinant bacteriophages and capture or the indicator protein or capture moiety-indicator protein fusion product to removed unbound, contaminating compounds.

In some embodiments, detection of the microorganism of interest may be completed without the need for culturing the sample as a way to increase the population of the microorganisms. For example, in certain embodiments the total time required for detection is less than 28.0 hours, 27.0 hours, 26.0 hours, 25.0 hours, 24.0 hours, 23.0 hours, 22.0 hours, 21.0 hours, 20.0 hours, 19.0 hours, 18.0 hours, 17.0 hours, 16.0 hours, 15.0 hours, 14.0 hours, 13.0 hours, 12.0 hours, 11.0 hours, 10.0 hours, 9.0 hours, 8.0 hours, 7.0 hours, 6.0 hours, 5.0 hours, 4.0 hours, 3.0 hours, 2.5 hours, 2.0 hours, 1.5 hours, or less than 1.0 hour. Minimizing time to result is critical in diagnostic testing.

In contrast to assays known in the art, the method of the disclosure can detect individual microorganisms. Thus, in certain embodiments, the method may detect as few as 10 cells of the microorganism present in a sample. For example, in certain embodiments, the recombinant indicator bacteriophage is highly specific for Staphylococcus spp., E. coli strains, Shigella spp., Klebsiella spp., Cutibacterium acnes, Proteus mirabalis, Enterococcus spp., or Pseudomonas spp. In an embodiment, the recombinant indicator bacteriophage can distinguish a bacterium of interest in the presence of other types of bacteria. In certain embodiments, the recombinant bacteriophage can be used to detect a single bacterium of the specific type in the sample. In certain embodiments, the recombinant indicator bacteriophage detects as few as 2, 3, 4, 5, 6, 7, 8, 9, 10, 15, 20, 30, 40, 50, 60, 70, 80, 90, or 100 of the specific bacteria in the sample. In further embodiments, the recombinant indicator bacteriophage assay may be used to quantify the number of viable microorganisms of interest present in the sample.

Thus, aspects of the present disclosure provide methods for detection of microorganisms in a test sample via an indicator protein product. In some embodiments, where the microorganism of interest is a bacterium, the indicator protein product may be associated with an infectious agent such as an indicator bacteriophage. The indicator protein product may react with a substrate to emit a detectable signal or may emit an intrinsic signal (e.g., bioluminescent protein). In some embodiments, the detection sensitivity can reveal the presence of as few as 50, 20, 10, 9, 8, 7, 6, 5, 4, 3, or 2 cells of the microorganism of interest in a test sample. In some embodiments, even a single cell of the microorganism of interest may yield a detectable signal.

In some embodiments, the indicator phage is genetically engineered to contain the gene for an enzyme, such as a luciferase, which is only produced upon infection of bacteria that the phage specifically recognizes and infects. In some embodiments, the indicator moiety is expressed late in the viral life cycle. In some embodiments, as described herein, the indicator is a soluble protein (e.g., soluble luciferase) and is not fused with a phage structural protein that limits its copy number.

In some embodiments, aliquots of a test sample comprising bacteria may be applied to a spin column and after infection with a recombinant bacteriophage and an optional washing to remove any excess bacteriophage, the amount of soluble indicator detected will be proportional to the amount of bacteriophage that are produced by infected bacteria.

Soluble indicator (e.g., luciferase) released into the surrounding liquid upon lysis of the bacteria may then be measured and quantified. In an embodiment, the solution is spun through the filter, and the filtrate collected for assay in a new receptacle (e.g., in a luminometer) following addition of a substrate for the indicator enzyme (e.g., luciferase substrate).

In various embodiments, the purified phage stock indicator phage does not comprise the detectable indicator itself, because the parental phage can be purified before it is used for incubation with a test sample. Expression of late (class III) genes occurs late in the viral life cycle. In some embodiments of the present invention, parental phage may be purified to exclude any existing indicator protein (e.g., luciferase). In some embodiments, expression of the indicator gene during bacteriophage replication following infection of host bacteria results in a soluble indicator protein product. In some embodiments, the soluble, indicator protein product is fused to a capture moiety (e.g., a peptide tag). In many embodiments, it is not necessary to separate parental from progeny phage prior to the detecting step. In an embodiment, the microorganism is a bacterium and the indicator phage is a bacteriophage. In an embodiment, the indicator protein product is a free, soluble luciferase, which is released upon lysis of the host microorganism. In some embodiments, the free, soluble luciferase is fused to a capture moiety (e.g., a peptide tag). For example, a capture moiety may be a peptide tag, such as an HA tag.

The assay may be performed in a variety of ways. In one embodiment, the sample is, incubated with phage, lysed, and then added to a 96-well plate coated with a binding partner, incubated with substrate, and then read. In other embodiments, the sample is added to a 96-well filter plate, the plate is centrifuged and media is added to bacteria collected on the filter before being incubated with phage. In still other embodiments, the sample is captured on at least one well of a 96-well plate using antibodies and washed with media to remove excess cells before being incubated with phage.

In another embodiments, the sample is incubated with phage, lysed, and then added to a lateral flow strip comprising a binding partner, incubated with substrate, and then read.

In some embodiments, lysis of the bacterium may occur before or during the detection step. Experiments suggest that infected unlysed cells may be detectable upon addition of luciferase substrate in some embodiments. Presumably, luciferase may exit cells and/or luciferase substrate may enter cells without complete cell lysis. For example, in some embodiments the substrate for the luciferase is cell-permeable (e.g., furimazine). Thus, for embodiments utilizing the spin filter system, where only luciferase released into the lysate (and not luciferase still inside intact bacteria) is analyzed in the luminometer, lysis is required for detection. However, for embodiments utilizing filter plates or 96-well plates with sample in solution or suspension, where the original plate full of intact and lysed cells is directly assayed in the luminometer, lysis is not necessary for detection.

In some embodiments, the reaction of indicator protein (e.g., luciferase) with substrate may continue for 60 minutes or more, and detection at various time points may be desirable for optimizing sensitivity. For example, in embodiments using 96-well filter plates as the solid support and luciferase as the indicator, luminometer readings may be taken initially and at 10- or 15-minute intervals until the reaction is completed.

Surprisingly, high concentrations of phage utilized for infecting test samples have successfully achieved detection of very low numbers of a target microorganism in a very short timeframe. The incubation of phage with a test sample in some embodiments need only be long enough for a single phage life cycle. In some embodiments, the bacteriophage concentration for this incubating step is greater than 1.0×10⁶, 2.0×10⁶, 3.0 10⁶, 5.0×10⁶, 6.0×10⁶, 7.0×10⁶, 8.0×10⁶, 9.0×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 3.0×10⁷, 4.0×10⁷, 5.0×10⁷, 6.0×10⁷, 7.0×10⁷, 8.0×10⁷, 9.0×10⁷, or 1.0×10⁸ PFU/mL.

Success with such high concentrations of phage is surprising because the large numbers of phage were previously associated with “lysis from without,” which killed target cells and thereby prevented generation of useful signal from earlier phage assays. It is possible that the clean-up of prepared phage stocks described herein helps to alleviate this problem (e.g., clean-up by sucrose gradient or cesium chloride isopycnic density gradient ultracentrifugation), because in addition to removing any contaminating luciferase associated with the phage, this clean-up may also remove ghost particles (particles that have lost DNA). The ghost particles can lyse bacterial cells via “lysis from without,” killing the cells prematurely and thereby preventing generation of indicator signal. Electron microscopy demonstrates that a crude phage lysate (i.e., before cesium chloride clean-up) may have greater than 50% ghosts. These ghost particles may contribute to premature death of the microorganism through the action of many phage particles puncturing the cell membrane. Thus ghost particles may have contributed to previous problems where high PFU concentrations were reported to be detrimental. Moreover, a very clean phage prep allows the assay to be performed with no wash steps, which makes the assay possible to perform without an initial concentration step. Some embodiments do include an initial concentration step, and in some embodiments this concentration step allows a shorter enrichment incubation time.

Some embodiments of testing methods may further include confirmatory assays. A variety of assays are known in the art for confirming an initial result, usually at a later point in time. For example, the samples can be cultured (e.g., selective chromogenic plating), and PCR can be utilized to confirm the presence of the microbial DNA, or other confirmatory assays can be used to confirm the initial result.

In certain embodiments, the methods of the present disclosure combine the use of a binding agent (e.g., antibody) to purify and/or concentrate a microorganism of interest such as Staphylococcus spp. from the sample in addition to detection with an infectious agent. For example, in certain embodiments, the invention comprises a method for detecting a microorganism of interest in a sample comprising the steps of: capturing the microorganism from the sample on a prior support using a capture antibody specific to the microorganism of interest such as Staphylococcus spp.; incubating the sample with a recombinant bacteriophage that infects Staphylococcus spp. wherein the recombinant bacteriophage comprises an indicator gene inserted into a late gene region of the bacteriophage such that expression of the indicator gene during bacteriophage replication following infection of host bacteria results in a soluble indicator protein product; and detecting the indicator protein product, wherein positive detection of the indicator protein product indicates that Staphylococcus spp. is present in the sample.

In some embodiments, synthetic phages are designed to optimize desirable traits for use in pathogen detection assays. In some embodiments, bioinformatics and previous analyses of genetic modifications are employed to optimize desirable traits. For example, in some embodiments, the genes encoding phage tail proteins can be optimized to recognize and bind to particular species of bacteria. In other embodiments the genes encoding phage tail proteins can be optimized to recognize and bind to an entire genus of bacteria, or a particular group of species within a genus. In this way, the phage can be optimized to detect broader or narrower groups of pathogens. In some embodiments, the synthetic phage may be designed to improve expression of the indicator gene. Additionally and/or alternatively, in some instances, the synthetic phage may be designed to increase the burst size of the phage to improve detection.

In some embodiments, the stability of the phage may be optimized to improve shelf-life. For example, enzybiotic solubility may be increased in order to increase subsequent phage stability. Additionally and/or alternatively phage thermostability may be optimized. Thermostable phage better preserve functional activity during storage thereby increasing shelf-life. Thus, in some embodiments, the thermostability and/or pH tolerance may be optimized.

### Determining Antibiotic Susceptibility

In order to determine antibiotic susceptibility of a particular bacteria of interest using the compositions and methods described herein, prior to incubation with the indicator cocktail composition, the sample is first incubated with and without an antibiotic.

The antibiotic referred to in this application can be any agent that is bacteriostatic (capable of inhibiting the growth of a microorganism) or bactericidal (capable of killing a microorganism). Thus, in certain embodiments, the methods may comprise detection of resistance of a microorganism of interest in a sample to an antibiotic by contacting the sample with the antibiotic, and incubating the sample that has been contacted with antibiotic with a recombinant bacteriophage that infects the microorganism of interest. This is distinct from those assays that detect the presence of genes (e.g., PCR) or proteins (e.g., antibody) that may confer antibiotic resistance, but do not test their functionality. Thus the current assay allows for phenotypic detection as opposed to genotypic detection.

In some instances, the bacteria of interest is not isolated from the sample prior to testing for antibiotic resistance. In certain embodiments, the sample is an uncultured or unenriched sample. In some cases, the method of detecting antibiotic resistance can be completed within 5 hours. In some embodiments, the method comprises treatment with lysis buffer to lyse the bacteria infected with the infectious agent prior to detecting the indicator protein.

The methods disclosed herein can be used to detect whether a microorganism of interest is susceptible or resistant to an antibiotic. A particular antibiotic may be specific for the type of microorganism it kills or inhibits; the antibiotic kills or inhibits the growth of microorganisms that are sensitive to the antibiotic and does not kill or inhibit the growth of microorganisms that are resistant to the antibiotic. In some cases, a previously sensitive microbial strain may become resistant. Resistance of microorganisms to antibiotics can be mediated by a number of different mechanisms. For example, some antibiotics disturb cell wall synthesis in a microorganism; resistance against such antibiotics can be mediated by altering the target of the antibiotic, namely a cell wall protein. In some cases, bacteria create resistance to an antibiotic by producing compounds capable of inactivating the antibiotic before reaching the bacteria. For example, some bacteria produce beta-lactamase, which is capable of cleaving the beta-lactam of penicillin or/and carbapenems, thus, inactivating these antibiotics. In some cases, the antibiotic is removed from the cell before reaching the target by a specific pump. An example is the RND transporter. In some cases, some antibiotics act by binding to ribosomal RNA (rRNA) and inhibit protein biosynthesis in the microorganism. A microorganism resistant to such antibiotic may comprise a mutated rRNA having a reduced binding capability to the antibiotic but having an essentially normal function within the ribosome. In other cases, bacteria harbor a gene that is capable of conferring resistance. For example, some MRSA harbor the mecA gene. The mecA gene product is an alternative transpeptidase with a low affinity for the ring-like structure of certain antibiotics which typically bind to transpeptidases required for bacterium cell wall formation. Therefore, antibiotics, including beta-lactams, are unable to inhibit cell wall synthesis in these bacteria. Some bacteria harbor antibiotic resistance genes that are non-functional, possibly due to mutation of the gene or regulation, which may be falsely detected as antibiotic-resistant with conventional nucleic acid methods, such as PCR, but not detected by functional methods, such as plating or culturing with antibiotics or this method.

Non-limiting examples of antibiotics that can be used in the invention include aminoglycosides, carbacephems, carbapenems, cephalosporins, glycopeptides, macrolides, monobactams, penicillin, beta-lactam antibiotic, quinolones, bacitracin, sulfonamides, tetracyclines, streptogramines, chloramphenicol, clindamycin, and lincosamide, cephamycins, lincomycins, daptomycin, oxazolidinone, and glycopeptide antibiotic.

As noted herein, in certain embodiments, the invention may comprise methods of using infectious particles for detecting resistance of microorganisms to an antibiotic or, stated another way, for detecting the efficacy of an antibiotic against a microorganism. In another embodiment, the invention comprises methods for selecting an antibiotic for treatment of an infection. Additionally, the methods may comprise methods for detecting antibiotic-resistant bacteria in a sample. The methods of the invention may be embodied in a variety of ways.

The method may comprise contacting the sample comprising the microorganism with the antibiotic and an infectious agent as described above. In some embodiments, the disclosure provides a method of determining effective dose of an antibiotic in killing or inhibiting the growth of a bacteria of interest comprising: (a) incubating each of one or more of antibiotic solutions separately with one or more samples comprising the microorganism, wherein the concentrations of the one or more of antibiotic solutions are different and define a range, (b) incubating the bacteria in the one or more of samples with a recombinant bacteriophage comprising an indicator gene, and wherein the recombinant bacteriophage is specific for the bacteria of interest, and (c) detecting an indicator protein product produced by the recombinant bacteriophage in the one or more of samples, wherein detection of the indicator protein product in one or more of the plurality of samples indicates the concentrations of antibiotic solutions used to treat the one or more of the one or more of samples are not effective, and the lack of detection of the indicator protein indicates the antibiotic is effective, thereby determining the effective dose of the antibiotic.

In other embodiments, the antibiotic and the infectious agent are added sequentially, e.g., the sample is contacted with the antibiotic before the sample is contacted with the infectious agent. In certain embodiments, the method may comprise incubating the sample with the antibiotic for a period time before contacting the sample with the infectious agent. The incubation time may vary depending on the nature of the antibiotic and the microorganism, for example based on the doubling time of the microorganism. In some embodiments, the incubation time is less than 24 hours, less than 18 hours, less than 12 hours, less than 6 hours, less than 5 hours, less than 4 hours, less than 3 hours, less than 2 hours, less than 1 hour, less than 45 min, or less than 30 min. The incubation time of microorganism with the infectious agent may also vary depending on the life cycle of the particular infectious agent, in some cases, the incubation time is less than 4 hours, less than 3 hours, less than 2 hours, less than 1 hour, less than 45 min, less than 30 min. Microorganisms that are resistant to the antibiotic will survive and may multiply, and the infectious agent that is specific to the microorganism will replicate resulting in production of the indicator protein product (e.g., luciferase); conversely, microorganisms that are sensitive to the antibiotic will be killed and thus the infectious agent will not replicate. Additionally, bacteriostatic antibiotics will not kill the bacteria; however, they will halt growth and/or enrichment of the bacteria. In some instances, bacteriostatic antibiotics may interfere with bacterial protein synthesis and are expected to prevent the bacteriophage from producing an indicator molecule (e.g., luciferase). The infectious agent according to this method comprises an indicator protein, the amount of which corresponds to the amount of the microorganisms present in the sample that have been treated with the antibiotic. Accordingly, a positive detection of the indicator protein indicates the microorganism is resistant to the antibiotic.

In some embodiments, the methods may be used to determine whether an antibiotic-resistant microorganism is present in a clinical sample. For example, the methods may be used to determine whether a patient is infected with Staphylococcus aureus that are resistant or susceptible to a particular antibiotic. A clinical sample obtained from a patient may then be incubated with an antibiotic specific for S. aureus. The sample may then be incubated with recombinant phage specific for S. aureus for a period of time. In samples with S. aureus resistant to the antibiotic, detection of the indicator protein produced by the recombinant phage will be positive. In samples with S. aureus susceptible to the antibiotic, detection of the indicator protein will be negative. In some embodiments, methods for detection of antibiotic resistance may be used to select an effective therapeutic to which the pathogenic bacterium is susceptible.

In certain embodiments the total time required for detection is less than 6.0 hours, 5.0 hours, 4.0 hours, 3.0 hours, 2.5 hours, 2.0 hours, 1.5 hours, or less than 1.0 hour. The total time required for detection will depend on the bacteria of interest, the type of phage, and antibiotic being tested.

Optionally, the method further comprises lysing the microorganism before detecting the indicator moiety. Any solution that does not affect the activity of the luciferase can be used to lyse the cells. In some cases, the lysis buffer may contain non-ionic detergents, chelating agents, enzymes or proprietary combinations of various salts and agents. Lysis buffers are also commercially available from Promega, Sigma-Aldrich, or Thermo-Fisher. Experiments suggest that infected unlysed cells may be detectable upon addition of luciferase substrate in some embodiments. Presumably, luciferase may exit cells and/or luciferase substrate may enter cells without complete cell lysis. For example, in some embodiments the substrate for the luciferase in cell-permeable (e.g., furimazine). Thus, for embodiments utilizing the spin filter system, where only luciferase released into the lysate (and not luciferase still inside intact bacteria) is analyzed in the luminometer, lysis is required for detection. However, for embodiments utilizing filter plates or 96-well plates with phage-infected sample in solution or suspension as described below, where intact and lysed cells may be directly assayed in the luminometer, lysis may not be necessary for detection. Thus, in some embodiments, the method of detecting antibiotic resistance does not involve lysing the microorganism.

A surprising aspect of embodiments of the assays is that the step of incubating the microorganism in a sample with infectious agent only needs to be long enough for a single life cycle of the infectious agent, e.g., a phage. The amplification power of using phage was previously thought to require more time, such that the phage would replicate for several cycles. A single replication of indicator phage may be sufficient to facilitate sensitive and rapid detection according to some embodiments of the present invention. Another surprising aspect of the embodiments of the assays is that high concentrations of phage utilized for infecting test samples (i.e., high MOI) have successfully achieved detection of very low numbers of antibiotic resistant target microorganisms that have been treated with antibiotic. Factors, including the burst size of the phage, can affect the number of phage life cycles, and therefore, amount of time needed for detection. Phage with a large burst size (approximately 100 PFU) may only require one cycle for detection, whereas phage with a smaller burst size (e.g., 10 PFU) may require multiple phage cycles for detection. In some embodiments, the incubation of phage with a test sample need only be long enough for a single phage life cycle. In other embodiments, the incubation of phage with a test sample is for an amount of time greater than a single life cycle. The phage concentration for the incubating step will vary depending on the type of phage used. In some embodiments, the phage concentration for this incubating step is greater than 1.0×10⁶, 2.0×10⁶, 3.0 10⁶, 5.0×10⁶, 6.0×10⁶, 7.0×10⁶, 8.0×10⁶, 9.0×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 3.0×10⁷, 4.0×10⁷, 5.0×10⁷, 6.0×10⁷, 7.0×10⁷, 8.0×10⁷, 9.0×10⁷, or 1.0×10⁸ PFU/mL. Success with such high concentrations of phage is surprising because such large numbers of phage were previously associated with “lysis from without,” which killed target cells immediately and thereby prevented generation of useful signal from earlier phage assays. It is possible that the purification of the phage stock described herein helps to alleviate this problem (e.g., purification by sucrose gradient cesium chloride isopycnic density gradient ultracentrifugation), because in addition to removing any contaminating luciferase associated with the phage, this purification may also remove ghost particles (particles that have lost DNA). The ghost particles can lyse bacterial cells via “lysis from without,” killing the cells prematurely and thereby preventing generation of indicator signal. Electron microscopy demonstrates that a crude recombinant phage lysate (i.e., before cesium chloride purification) may have greater than 50% ghosts. These ghost particles may contribute to premature death of the microorganism through the action of many phage particles puncturing the cell membrane. Thus ghost particles may have contributed to previous problems where high PFU concentrations were reported to be detrimental.

Any of the indicator moieties as described in this disclosure may be used for detecting the viability of microorganisms after antibiotic treatment, thereby detecting antibiotic resistance. In some embodiments, the indicator moiety associated with the infectious agent may be detectable during or after replication of the infectious agent. For example, as described above, in some cases, the indicator moiety may be a protein that emits an intrinsic signal, such as a fluorescent protein (e.g., green fluorescent protein or others). The indicator may generate light and/or may be detectable by a color change. In some embodiments, a luminometer may be used to detect the indicator (e.g., luciferase). However, other machines or devices may also be used. For example, a spectrophotometer, CCD camera, or CMOS camera may detect color changes and other light emissions.

In some embodiments, exposure of the sample to antibiotic may continue for 5 minutes or more and detection at various time points may be desirable for optimal sensitivity. For example, aliquots of a primary sample treated with antibiotic can be taken at different time intervals (e.g., at 5 minutes, 10 minutes, or 15 minutes). Samples from varying time interval may then be infected with phage and indicator protein measured following the addition of substrate.

In some embodiments, detection of the signal is used to determine antibiotic resistance. In some embodiments, the signal produced by the sample is compared to an experimentally determined value. In further embodiments, the experimentally determined value is a signal produced by a control sample. In some embodiments, the background threshold value is determined using a control without microorganisms. In some embodiments, a control without phage or without antibiotic, or other control samples may also be used to determine an appropriate threshold value. In some embodiments, the experimentally determined value is a background threshold value calculated from an average background signal plus standard deviation of 1-3 times the average background signal, or greater. In some embodiments, the background threshold value may be calculated from average background signal plus standard deviation of 2 times the average background signal. In other embodiments, the background threshold value may be calculated from the average background signal times some multiple (e.g., 2 or 3). Detection of a sample signal greater than the background threshold value indicates the presence of one or more antibiotic-resistant microorganisms in the sample. For example, the average background signal may be 250 RLU. The threshold background value may be calculated by multiplying the average background signal (e.g., 250) by 3 to calculate a value of 750 RLU. Samples with bacteria having a signal value greater than 750 RLU are determined to be positive for containing antibiotic-resistant bacteria.

Alternatively, the experimentally determined value is the signal produced by a control sample. Assays may include various appropriate control samples. For example, samples containing no infectious agent that is specific to the microorganism, or samples containing infectious agents but without microorganism, may be assayed as controls for background signal levels. In some cases, samples containing the microorganisms that have not been treated with the antibiotic, are assayed as controls for determining antibiotic resistance using the infectious agents.

In some embodiments, the sample signal is compared to the control signal to determine whether antibiotic-resistant microorganisms are present in the sample. Unchanged detection of the signal as compared to a control sample that is contacted with the infectious agent but not with the antibiotic indicates the microorganism is resistant to the antibiotic, and reduced detection of the indicator protein as compared to a control sample that is contacted with infectious agent but not with antibiotic indicates the microorganism is susceptible to the antibiotic. Unchanged detection refers to the detected signal from a sample that has been treated with the antibiotic and infectious agent is at least 80%, at least 90%, or at least 95% of signal from a control sample that has not been treated with the antibiotic. Reduced detection refers to the detected signal from a sample that has been treated with the antibiotic and infectious agent is less than 80%, less than 70%, less than 60%, less than 50%, less than 40%, or at least 30% of signal from a control sample that has not been treated with the antibiotic.

Optionally, the sample comprising the microorganism of interest is an uncultured sample. Features of each of the compositions used in the methods, as described above, can be also be utilized in the methods for detecting antibiotic resistance of the microorganism of interest.

Also provided herein is a method of determining the effective dose of an antibiotic for killing a microorganism. In some embodiments, the antibiotic is effective at killing Staphylococcus species. For example, the antibiotic may be cefoxitin, which is effective against most methicillin-sensitive S. aureus (MSSA). Typically, one or more antibiotic solutions having different concentrations are prepared such that the different concentrations of the solutions define a range. In some cases, the concentration ratio of the least concentrated antibiotic solution to the most concentrated antibiotic solution ranges from 1:2 to 1:50, e.g., from 1:5 to 1:30, or from 1:10 to 1:20. In some cases, the lowest concentration of the one or more antibiotic solution is at least 1 μg/mL, e.g., at least 2 μg/mL, at least 5 μg/mL at least 10 μg/mL, at least 20 μg/mL, at least 40 μg/mL, at least 80 μg/mL, or at least 100 μg/mL. Each of the one or more antibiotic solutions is incubated with one aliquot of the sample comprising the microorganism of interest. In some cases, the infectious agent (e.g., bacteriophage) that is specific to the microorganism is added simultaneously with the antibiotic solutions. In some cases, the aliquots of sample are incubated with the antibiotic solutions for a period of time before the addition of the infectious agent. The indicator protein product can be detected, and positive detection indicates that the antibiotic solution is not effective and negative detection indicates the antibiotic solution is effective. The concentration of the antibiotic solution is expected to correlate to an effective clinical dose. Accordingly, in some embodiments, the method of determining effective dose of an antibiotic in killing a microorganism of interest comprises incubating each of one or more antibiotic solutions separately with a microorganism of interest in a sample, wherein the concentrations of the one or more antibiotic solutions are different and define a range; incubating the microorganism in the one or more samples with an infectious agent comprising an indicator moiety; detecting the indicator protein product of the infectious agent in the one or more samples, wherein positive detection of the indicator protein product in one or more of the one or more samples indicates the concentrations of antibiotic solutions used to treat the one or more of the one or more samples are not effective, and the lack of detection of the indicator protein indicates the antibiotic is effective, thereby determining the effective dose of the antibiotic.

In some embodiments, the method allows for determination of categorical assignment for antibiotic resistance. For example, the method disclosed herein may be used to determine the categorical assignment (e.g., susceptible, intermediate, and resistant) of an antibiotic. Susceptible antibiotics are those that are likely, but not guaranteed to inhibit the pathogenic microbe; may be an appropriate choice for treatment. Intermediate antibiotics are those that may be effective at a higher dosage, or more frequent dosage, or effective only in specific body sites where the antibiotic penetrates to provide adequate concentrations. Resistant antibiotics are those that are not effective at inhibiting the growth of the organism in a laboratory test; may not be an appropriate choice for treatment. In some embodiments, two or more antibiotic solutions are tested and the concentration ratio of the least concentrated solution and the most concentrated solution in the one or more antibiotic solutions ranges from 1:2 to 1:50, e.g., from 1:5 to 1:30, or from 1:10 to 1:20. In some cases, the lowest concentration of the one or more antibiotic solution is at least 1 μg/mL, e.g., at least 2 μg/mL, at least 5 μg/mL at least 10 μg/mL, at least 20 μg/mL, at least 40 μg/mL, at least 80 μg/mL, or at least 100 μg/mL.

In some embodiments, the present invention comprises methods for detecting antibiotic-resistant microorganisms in the presence of antibiotic-sensitive microorganisms. In certain instances, detection of antibiotic-resistant bacteria can be used to prevent the spread of infection in healthcare settings. Preventative measures may then be implemented to prevent the spread of antibiotic-resistant bacteria.

In some embodiments of methods for detecting antibiotic resistant microorganisms, samples may contain both antibiotic-resistant and antibiotic-sensitive bacteria. For example, samples may comprise both MRSA and MSSA. In some embodiments, MRSA can be detected in the presence of MSSA without the need for isolation of MRSA from the sample. In the presence of antibiotic, MSSA does not generate a signal above the threshold value, but MRSA present in the sample are capable of producing a signal above the threshold value. Thus, if both are present within a sample, a signal above the threshold value indicates the presence of an antibiotic-resistant strain (e.g. MRSA).

In contrast to many assays known in the art, detection of antibiotic resistance of a microorganism can be achieved without prior isolation. Many methods require that a sample is cultured beforehand to purify/isolate individual colonies of the bacterium on an agar plate. The increased sensitivity of the methods disclosed herein, is due in part to the ability of a large number of specific infectious agents, e.g., phages to bind to a single microorganism. Following infection and replication of the phage, target microorganisms may be detected via an indicator protein product produced during phage replication.

Thus, in certain embodiments, the method may detect antibiotic resistance of a microorganism in a sample that comprises ≤10 cells of the microorganism (i.e., 1, 2, 3, 4, 5, 6, 7, 8, 9, 10 microorganisms). In certain embodiments, the recombinant phage can be used to detect antibiotic resistance by detection of a single bacterium of the specific type in the sample that has been treated with the antibiotic. In certain embodiments, the recombinant phage detects the presence of as few as 2, 3, 4, 5, 6, 7, 8, 9, 10, 15, 20, 30, 40, 50, 60, 70, 80, 90, or 100 of the specific bacteria in the sample that has been contacted with antibiotic.

The sensitivity of the method of detecting antibiotic resistance as disclosed herein may be further increased by washing the captured and infected microorganisms prior to incubation with the antibiotic. Isolation of target bacteria may be required when the antibiotic being assessed is known to be degraded by other bacterial species. For example, penicillin resistance would be difficult to assess without purification, since other bacteria present in a sample could degrade the antibiotic (beta-lactamase secretion) and lead to a false positive. Additionally, captured microorganisms may be washed following incubation with antibiotic and the infectious agent, prior to addition of lysis buffer and substrate. These additional washing steps aid in the removal of excess parental phage and/or luciferase or other indicator protein contaminating the phage preparation. Accordingly, in some embodiments, the method of the detecting antibiotic resistance may comprise washing the captured and infected microorganisms, after adding the phage but before incubating.

Thus, some embodiments of the present invention solve a need by using infectious agent-based methods for amplifying a detectable signal, thereby indicating whether a microorganism is resistant to an antibiotic. The invention allows a user to detect antibiotic resistance of a microorganism that is present in a sample has not been purified or isolated. In certain embodiments as little as a single bacterium is detected. This principle allows amplification of indicator signal from one or a few cells based on specific recognition of microorganism surface receptors. For example, by exposing even a single cell of a microorganism to a plurality of phage, thereafter allowing amplification of the phage and high-level expression of an encoded indicator gene product during replication, the indicator signal is amplified such that the single microorganism is detectable. The present invention excels as a rapid test for the detection of microorganisms by not requiring isolation of the microorganisms prior to detection. In some embodiments detection is possible within 1-2 replication cycles of the phage or virus.

### Systems and Kits of the Invention

In some embodiments, the disclosure comprises systems (e.g., automated systems or kits) comprising components for performing the methods disclosed herein. In some embodiments, indicator phage are comprised in systems or kits according to the invention. Methods described herein may also utilize such indicator phage systems or kits. Some embodiments described herein are particularly suitable for automation and/or kits, given the minimal amount of reagents and materials required to perform the methods. In certain embodiments, each of the components of a kit may comprise a self-contained unit that is deliverable from a first site to a second site.

In some embodiments, the disclosure comprises systems or kits for rapid detection of a microorganism of interest in a sample. In one aspect the kit for detecting a bacteria of interest comprises: (i) recombinant bacteriophage comprising a genetic construct inserted into the bacteriophage genome, wherein the genetic construct comprises an indicator gene encoding an indicator protein product; and (ii) a surface for capturing the indicator protein product, wherein the surface comprises a binding partner. In some embodiments, the kit further comprises a detection reagent. In some embodiments, the kit further comprises a lysis buffer.

The systems or kits may in certain embodiments comprise a component for incubating the sample, one or more indicator bacteriophage compositions as described in detail herein; a component for capturing the indicator protein as described in detail herein, and a component for detecting the indicator protein product.

In other embodiments, the disclosure comprises a system or kit for rapid detection of a microorganism of interest in a sample, comprising one or more indictor cocktail compositions. As described herein, each indicator cocktail composition is specific for a bacteria of interest, wherein the recombinant bacteriophage comprises a genetic construct, and wherein the genetic construct comprises a gene encoding an indicator protein product. In some embodiments, the genetic construct further comprises a capture moiety and an exogenous promoter.

In certain embodiments, each indicator cocktail compositions is highly specific for a particular bacterium. In an embodiment, the recombinant bacteriophage can distinguish the bacterium of interest in the presence of more than 100 other types of bacteria. In certain embodiments, a system or kit detects a single bacterium of the specific type in the sample. In certain embodiments, a system or kit detects and quantifies as few as 2, 3, 4, 5, 6, 7, 8, 9, 10, 15, 20, 30, 40, 50, 60, 70, 80, 90, or 100 specific viable bacteria in the sample.

In some embodiments, the systems and/or kits may further comprise a component for collecting the microorganism of interest. In some embodiments, samples may be collected using swabs of solid surfaces (e.g., medical devices or food processing equipment). Thus, in some embodiments, the systems and/or kits may further comprise a swab. In other embodiments, samples may be collected using irrigation may be used to collect the sample. Irrigation is the flow of a solution (e.g., saline) across a surface. Thus, in some embodiments, the systems and/or kits may further comprise an irrigant solution (e.g., saline).

In some embodiments, the systems and kits further comprise a surface for capturing the indicator protein product as described in detail above. For example, the surface may be a microtiter plate, latex particle, lateral flow strip, bead, magnetic particle, dipstick, microsphere or any other surface capable of capturing the indicator protein product. In some embodiments the surface comprises a capture area comprising an immobilized binding partner. In some embodiments, the binding partner is specific for the indicator protein product. In other embodiments, the binding partner is specific for the capture moiety fused to the indicator protein.

In certain embodiments, the systems and/or kits may further comprise a component for washing the captured sample. Additionally or alternatively, the systems and/or kits may further comprise a component for determining amount of the indicator protein product, wherein the amount of indicator protein product detected corresponds to the amount of microorganism in the sample. For example, in certain embodiments, the system or kit may comprise a luminometer or other device for measuring a luciferase enzyme activity. In some embodiments, the luminometer is a handheld device.

In some systems and/or kits, the same component may be used for multiple steps. In some systems and/or kits, the steps are automated or controlled by the user via computer input and/or wherein a liquid-handling robot performs at least one step.

Thus in certain embodiments, the invention may comprise a system or kit for rapid detection of a microorganism of interest in a sample, comprising: a component for incubating the sample with a recombinant bacteriophage specific for the microorganism of interest, wherein the recombinant bacteriophage comprises a gene encoding an indicator protein product; a component for capturing the microorganism from the sample on a solid support; a component for washing the captured microorganism sample to remove unbound infectious agent; and a component for detecting the indicator protein product. In some embodiments, the same component may be used for steps of capturing and/or incubating and/or washing (e.g., a filter component). Some embodiments additionally comprise a component for determining the amount of the microorganism of interest in the sample, wherein the amount of indicator protein product detected corresponds to the amount of microorganism in the sample. Such systems can include various embodiments and subembodiments analogous to those described above for methods of rapid detection of microorganisms. In an embodiment, the microorganism is a bacterium and the infectious agent is a bacteriophage. In a computerized system, the system may be fully automated, semi-automated, or directed by the user through a computer (or some combination thereof).

In an embodiment, the disclosure comprises a system or kit comprising components for detecting a microorganism of interest comprising: a component for infecting the at least one microorganism with a plurality of recombinant bacteriophages; a component for lysing the at least one infected microorganism; and a component for detecting the soluble indicator protein product encoded and expressed by the recombinant bacteriophage, wherein detection of the soluble protein product of the infectious agent indicates that the microorganism is present in the sample.

In some embodiments, the systems and kits of the disclosure further comprise an antibiotic for determining antibiotic susceptibility of the bacteria of interest.

These systems and kits of the disclosure include various components. As used herein, the term “component” is broadly defined and includes any suitable apparatus or collections of apparatuses suitable for carrying out the recited method. The components need not be integrally connected or situated with respect to each other in any particular way. The invention includes any suitable arrangements of the components with respect to each other. For example, the components need not be in the same room. But in some embodiments, the components are connected to each other in an integral unit. In some embodiments, the same components may perform multiple functions.

In one aspect, a system for detecting a bacteria of interest comprises: (a) a recombinant bacteriophage comprising a genetic construct inserted into the bacteriophage genome, wherein the recombinant bacteriophage comprises a genetic construct inserted into the bacteriophage genome, and wherein the genetic construct comprises an indicator gene and a capture moiety encoding a capture moiety-indicator protein fusion product; (b) a surface for capturing capture moiety-indicator protein fusion product, wherein the surface comprises a binding partner; and (c) a component for detecting the indicator protein product.

The system, as described in the present technique or any of its components, may be embodied in the form of a computer system. Typical examples of a computer system include a general-purpose computer, a programmed microprocessor, a microcontroller, a peripheral integrated circuit element, and other devices or arrangements of devices that are capable of implementing the steps that constitute the method of the present technique.

A computer system may comprise a computer, an input device, a display unit, and/or the Internet. The computer may further comprise a microprocessor. The microprocessor may be connected to a communication bus. The computer may also include a memory. The memory may include random access memory (RAM) and read only memory (ROM). The computer system may further comprise a storage device. The storage device can be a hard disk drive or a removable storage drive such as a floppy disk drive, optical disk drive, etc. The storage device can also be other similar means for loading computer programs or other instructions into the computer system. The computer system may also include a communication unit. The communication unit allows the computer to connect to other databases and the Internet through an I/O interface. The communication unit allows the transfer to, as well as reception of data from, other databases. The communication unit may include a modem, an Ethernet card, or any similar device which enables the computer system to connect to databases and networks such as LAN, MAN, WAN and the Internet. The computer system thus may facilitate inputs from a user through input device, accessible to the system through I/O interface.

A computing device typically will include an operating system that provides executable program instructions for the general administration and operation of that computing device, and typically will include a computer-readable storage medium (e.g., a hard disk, random access memory, read only memory, etc.) storing instructions that, when executed by a processor of the server, allow the computing device to perform its intended functions. Suitable implementations for the operating system and general functionality of the computing device are known or commercially available, and are readily implemented by persons having ordinary skill in the art, particularly in light of the disclosure herein.

The computer system executes a set of instructions that are stored in one or more storage elements, in order to process input data. The storage elements may also hold data or other information as desired. The storage element may be in the form of an information source or a physical memory element present in the processing machine.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers, or other network devices may be stored locally and/or remotely, as appropriate. Where a system includes computing devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or Web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

Non-transient storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system device. Based on the disclosure and teachings provided herein, a person of ordinary skill in the art will appreciate other ways and/or methods to implement the various embodiments.

A computer-readable medium may comprise, but is not limited to, an electronic, optical, magnetic, or other storage device capable of providing a processor with computer-readable instructions. Other examples include, but are not limited to, a floppy disk, CD-ROM, DVD, magnetic disk, memory chip, ROM, RAM, SRAM, DRAM, content-addressable memory (“CAM”), DDR, flash memory such as NAND flash or NOR flash, an ASIC, a configured processor, optical storage, magnetic tape or other magnetic storage, or any other medium from which a computer processor can read instructions. In one embodiment, the computing device may comprise a single type of computer-readable medium such as random access memory (RAM). In other embodiments, the computing device may comprise two or more types of computer-readable medium such as random access memory (RAM), a disk drive, and cache. The computing device may be in communication with one or more external computer-readable mediums such as an external hard disk drive or an external DVD or Blu-Ray drive.

As discussed above, the embodiment comprises a processor which is configured to execute computer-executable program instructions and/or to access information stored in memory. The instructions may comprise processor-specific instructions generated by a compiler and/or an interpreter from code written in any suitable computer-programming language including, for example, C, C++, C#, Visual Basic, Java, Python, Perl, JavaScript, and ActionScript (Adobe Systems, Mountain View, Calif.). In an embodiment, the computing device comprises a single processor. In other embodiments, the device comprises two or more processors. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

The computing device comprises a network interface. In some embodiments, the network interface is configured for communicating via wired or wireless communication links. For example, the network interface may allow for communication over networks via Ethernet, IEEE 802.11 (Wi-Fi), 802.16 (Wi-Max), Bluetooth, infrared, etc. As another example, network interface may allow for communication over networks such as CDMA, GSM, UMTS, or other cellular communication networks. In some embodiments, the network interface may allow for point-to-point connections with another device, such as via the Universal Serial Bus (USB), 1394 FireWire, serial or parallel connections, or similar interfaces. Some embodiments of suitable computing devices may comprise two or more network interfaces for communication over one or more networks. In some embodiments, the computing device may include a data store in addition to or in place of a network interface.

Some embodiments of suitable computing devices may comprise or be in communication with a number of external or internal devices such as a mouse, a CD-ROM, DVD, a keyboard, a display, audio speakers, one or more microphones, or any other input or output devices. For example, the computing device may be in communication with various user interface devices and a display. The display may use any suitable technology including, but not limited to, LCD, LED, CRT, and the like.

The set of instructions for execution by the computer system may include various commands that instruct the processing machine to perform specific tasks such as the steps that constitute the method of the present technique. The set of instructions may be in the form of a software program. Further, the software may be in the form of a collection of separate programs, a program module with a larger program or a portion of a program module, as in the present technique. The software may also include modular programming in the form of object-oriented programming. The processing of input data by the processing machine may be in response to user commands, results of previous processing, or a request made by another processing machine.

While the present invention has been disclosed with references to certain embodiments, numerous modifications, alterations and changes to the described embodiments are possible without departing from the scope and spirit of the present invention, as defined in the appended claims. Accordingly, it is intended that the present invention not be limited to the described embodiments, but that it have the full scope defined by the language of the following claims, and equivalents thereof.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

### Example 1. Direct Coating of Medium and High Protein Binding Plates with Anti-Indicator Protein Antibodies

Mouse anti-NANOLUC® luciferase purified monoclonal IgG antibody (MsXNanoluc) was reconstituted in 200 μL of PBS to produce a 0.5 mg/mL MsXNanoluc antibody solution, which was then diluted to 1 μg/mL in PBS. 100 μL was pipetted into each well of a medium (Greiner bio-one—Strips Plate 12xF8, PS, F-Bottom, White, Lumitrac, Med Binding, Ref #762075) or high (Greiner bio-one -High Binding Ref #762074) protein binding plate and incubated overnight at 2-8° C. for 22 hours. The plates were then washed with 300 of 1X PBS/well. Then the plates were blocked with 300 μL of 5% BSA in PBS for 2 hours at room temperature and then washed 3 times with 300 μL of PBS/well/wash. Then the plates were over-coated with 300 μL of 5% sucrose in PBS for one hour at room temperature. Then the overcoat was decanted and plates were stored in a container at 4-8° C. for long-term storage.

### Example 2. Detection of Staphylococcus aureus in Human Blood

S. aureus (ATCC 12600) was grown to log phase (OD600 of 0.41) in tryptic soy broth (TSB). Cultures were diluted in TSB to obtain the desired burden, which was confirmed by plating on TSB agar for colony forming units (CFU). 12.5 μL of each dilution was added directly to 37.5 μL of TSB or human blood in 96-well plates (High binding). When indicated, some plates contained bound anti-NANOLUC® antibody for capture. Human blood was collected from a single donor using sodium heparin as an anti-coagulant. For blood samples, 100 μL of TSB containing sodium polyanethole sulfonate (SPS) was added to achieve a 25% human blood matrix. The final concentration of SPS in the well (150 μL volume) was 0.05%. For TSB samples, 100 μL of TSB was added to achieve the same 150 μL volume. Test strips were then sealed with cover film and incubated at 37° C. for 30 minutes. After this brief enrichment, 20 μL of phage working stock was added to wells containing the TSB matrix. Phage working stock contained 8×10⁷ plaque forming units per mL of both MP115.NL and SAPJV1.NL. To permit infection in wells containing blood matrix, 0.5 mg of recombinant Staphylococcal protein A (pro-356, Prospec, Ness-Ziona, Israel) per well was included as indicated within the 20 μL of phage working stock. Assay strips were once again sealed with cover film and incubated at 37° C. for three hours. Following infection, these strips were washed three times with 300 μL PBS-T (10 mM sodium phosphate, 150 mM NaCl, 0.05% Tween 20, pH 7.4). Washes were conducted using an automatic plate washer (AccuWash, Thermo Fisher Scientific, Waltham, Mass., USA). 100 μL of NanoGlo buffer (Promega, Madison, Wisc., USA) containing 1 μL of NanoGlo substrate (Promega, Madison, Wisc., USA) was added to each well. Following a 3 minute wait period, the signal output of each sample as relative light units (RLU) was determined using a GloMax Navigator (Promega, Madison, Wisc., USA). Signal over background (SB) was calculated by dividing the RLU from each sample from the RLU observed in the media control for that test matrix (Table 1).

TABLE 1
Detection of Staphylococcus aureus in Human Blood

| | | | | | Anti-NanoLuc | | | |
|---|---|---|---|---|---|---|---|---|
| | | | Test | Protein A Added | Capture Strips | | Control Strips | |
| Sample | Burden | CFU/well | Matrix | During Infection | RLU | S/B | RLU | S/B |
| S. aureus | High | 5150 | TSB | No | 614600 | 15365 | 1100 | 65 |
| S. aureus | Low | 52 | TSB | No | 525 | 13 | 7 | 0 |
| Media Control | — | N/A | TSB | No | 40 | 1 | 17 | 1 |
| S. aureus | High | 5150 | Blood | No | 75 | 4 | 8 | 1 |
| S. aureus | Low | 52 | Blood | No | 17 | 1 | 7 | 1 |
| S. aureus | High | 5150 | Blood | Yes | 168800 | 8440 | 13 | 1 |
| S. aureus | Low | 52 | Blood | Yes | 562 | 28 | 8 | 1 |
| Media Control | — | N/A | Blood | Yes | 20 | 1 | 12 | 1 |

In these examples, the anti-NANOLUC® antibody is the immobilized binding partner. Table 1 demonstrates a substantial increase in signal detection when the indicator protein is captured by an immobilized binding partner. For example, in samples with a low burden or high burden of S. aureus, the RLU is significantly higher when the indicator protein is captured by the anti-NANOLUC® antibody than when the sample is not captured using the control strips. Surprisingly, no infection of the S. aureus can take place if the S. aureus has bound IgG. The addition of Protein A allows S. aureus to be infected. Red blood cells and other serum proteins do not interfere with the capture of expressed indicator protein. Additionally, quenching of the signal as seen in the control by the red blood cells is eliminated and signal over background is maintained or increased. Thus, the indicator protein can be detected using whole blood samples with minimal interference from other components in the sample (e.g., proteins). Conventionally, serum or plasma is isolated from the blood for reliable detection of the indicator protein product. Advantageously, the examples demonstrate that the methods of detection can be done on whole blood samples taken directly from a patient by using this capture step.

### Example 3. Antibiotic Susceptibility Testing in Human Blood

Methicillin-resistant Staphylococcus aureus (MRSA) strains (ATCC BAA-1720, CDC AR0480) and the methicillin-susceptible Staphylococcus aureus (MSSA) strain (ATCC 12600) were grown to log phase (OD600 ranged from 0.16 to 0.4) in tryptic soy broth (TSB). Cultures were diluted in TSB to obtain the desired burden, which was confirmed by plating on TSB agar for colony forming units (CFU). 50 μL of each dilution was added to test strips. When indicated, some strips contained bound anti-NANOLUC® antibody on medium-binding plates for capture. 85 μL, of either TSB or human blood diluted with TSB and sodium polyanethole sulfonate (SPS) was added. Human blood was collected from a single donor using sodium heparin as an anti-coagulant. Each well then received 15 μL of either TSB or 22 μg/mL cefoxitin (FOX) in TSB. The final concentration of each component in the well (150 μL volume) was 25% human blood, 0.0375% SPS, and 2.2 μg/mL FOX. Test strips were then sealed with cover film and incubated at 37° C. for two hours. After this selective enrichment, 20 μL of phage working stock was added to wells containing the TSB matrix. Phage working stock contained 8×10⁷ plaque forming units per mL (pfu/mL) of MP115.NL and 6.9×10⁸ pfu/mL of SAPJV1.NL. For wells containing blood matrix, 0.5 mg of recombinant Staphylococcal protein A (pro-356, Prospec, Ness-Ziona, Israel) per well was included within the 20 μL of phage working stock. Assay strips were once again sealed with cover film and incubated at 37° C. for three hours. Following infection, anti-NanoLuc capture and controls strips were washed three times with 300 μL PBS-T (10 mM sodium phosphate, 150 mM NaCl, 0.05% Tween 20, pH 7.4). Washes were conducted using an automatic plate washer (AccuWash, Thermo Fisher Scientific, Waltham, Mass., USA). 100 μL of NanoGlo buffer (Promega, Madison, Wisc., USA) containing 1 μL of NanoGlo substrate (Promega, Madison, Wisc., USA) was added to each well. “No wash+No Capture” strips were not washed and instead received 65 μL of a master mix containing 50 μL NanoGlo Buffer, 15 μL TSB, and 1 μL NanoGlo substrate. Following a 3 minute wait period, the signal output of each sample as relative light units (RLU) was determined using a GloMax Navigator (Promega, Madison, Wisc., USA). Signal over background (SB) was calculated by dividing the RLU from each sample from the RLU observed in the media control for that test matrix (Table 2).

TABLE 2
Antibiotic Susceptibility Testing in Human Blood

| | | | | |
|---|---|---|---|---|
| No Capture + No Wash | TSB | TSB + FOX | Blood | Blood + FOX |

| Sample | Susceptibility | CFU/Well | RLU | S/B | RLU | S/B | RLU | S/B | RLU | S/B |
|---|---|---|---|---|---|---|---|---|---|---|
| BAA-1720 (MRSA) | Resistant | 41 | 168400 | 208 | 342000 | 447 | 29440 | 775 | 9035 | 177 |
| AR0480 (MRSA) | Resistant | 110 | 631600 | 779 | 127000 | 166 | 36890 | 971 | 432 | 8 |
| 12600 (MSSA) | Susceptible | 130 | 393100 | 485 | 988 | 1 | 42810 | 1127 | 103 | 2 |
| Media (Control) | N/A | N/A | 811 | 1 | 765 | 1 | 38 | 1 | 51 | 1 |

| Anti-NanoLuc Capture Strips | | | TSB | | TSB + FOX | | Blood | | Blood + FOX | |
|---|---|---|---|---|---|---|---|---|---|---|
| Sample | Susceptibility | CFU/Well | RLU | S/B | RLU | S/B | RLU | S/B | RLU | S/B |
| BAA-1720 (MRSA) | Resistant | 41 | 53610 | 623 | 79180 | 1028 | 112900 | 6272 | 63960 | 3998 |
| AR0480 (MRSA) | Resistant | 110 | 137700 | 1601 | 96650 | 1255 | 286400 | 15911 | 6927 | 433 |
| 12600 (MSSA) | Susceptible | 130 | 95180 | 1107 | 80 | 1 | 330900 | 18383 | 18 | 1 |
| Media (Control) | N/A | N/A | 86 | 1 | 77 | 1 | 18 | 1 | 16 | 1 |

| 5% BSA Blocked Strips | | | TSB | | TSB + FOX | | Blood | | Blood + FOX | |
|---|---|---|---|---|---|---|---|---|---|---|
| Sample | Susceptibility | CFU/Well | RLU | S/B | RLU | S/B | RLU | S/B | RLU | S/B |
| BAA-1720 (MRSA) | Resistant | 41 | 210 | 35 | 20 | 3 | 21 | 2 | 12 | 2 |
| AR0480 (MRSA) | Resistant | 110 | 51 | 9 | 53 | 7 | 16 | 2 | 3 | 0 |
| 12600 (MSSA) | Susceptible | 130 | 120 | 20 | 6 | 1 | 15 | 2 | 10 | 1 |
| Media (Control) | N/A | N/A | 6 | 1 | 8 | 1 | 10 | 1 | 8 | 1 |

In Table 2, the examples for “No Capture+No Wash” demonstrated the total signal generated and the drop in signal due to cefoxitin when the assay is done in just media (TSB). When done in the presence of blood, the signal is quenched. When the capture strips are used, there is a substantial increase in signal due to removal of the quenching done by blood. The 5% BSA blocked strip (bovine serum albumin, Sigma Life Science Product #A9647) is to show non-specific binding. Once again, the examples demonstrate a substantial increase in signal detection when the indicator protein was captured by an immobilized binding partner for whole blood samples. Additionally, the signal detection was significantly improved by the capture step for whole blood samples that included an antibiotic. Surprisingly, the indicator protein can be detected using whole blood samples with minimal interference from other components in the sample.

### Example 4. Indirect Coating of Medium and High Protein Binding Plates with Anti-Indicator Protein Antibodies

Rabbit anti-mouse IgG (Abcam, Catalog #46540) or goat anti-mouse IgG (Abcam, Catalog #6708) were diluted in PBS to 10 μg/mL. 100 μL of diluted rabbit anti-mouse IgG or goat anti-mouse IgG was then pipetted into each well of a medium (Greiner bio-one—Strips Plate 12xF8, PS , F-Bottom, White, Lumitrac, Med Binding, Ref #762075) or high (Greiner bio-one—High Binding Ref #762074) protein binding plate and incubated overnight at 2-8° C. for 18-20 hours. The plates were then washed three times with 300 μL of 1X PBS/well/wash. Mouse anti-NANOLUC® luciferase purified monoclonal IgG antibody (MsXNANOLUC®) was diluted to 1 μg/mL in PBS. 100 μL was pipetted into each well coated with rabbit or goat anti-mouse IgG and incubated at 2-8° C. for 18-20 hours. The plates were then washed three times with 300 μL of 1X PBS/well/wash. Then the plates were blocked with 300 μL of 5% BSA in PBS for 2 hours at room temperature and then washed 3 times with 300 μL of PBS/well/wash. Then the plates were over-coated with 300 μL of 5% sucrose in PBS for one hour at room temperature. Then the overcoat was decanted and plates were dried overnight before being transferred to plastic bags and stored at 2-8° C.

### Example 5. Titration of NANOLUC® on Coated Plates

A stock solution of purified NANOLUC® at 1.5 mg/mL was diluted to 1 ng/mL in PBS. Serial 10 fold dilutions in PBS were made from the 1 ng/mL to 0.001 pg/mL. 100 μL of each dilution was transferred to a well of strips (medium and high binding) coated with MsxNANOLUC®, GtxMsIgG/MsxNANOLUC®, RbxMsIgG/MsxNANOLUC®. A 5% BSA blocked strip was included for NSB determination and an uncoated strip for NANOLUC® activity measurement. Assay strips were sealed with cover film and incubated at 37° C. for three hours. Antibody coated strips were washed three times with 300 μL/well PBS-T (10 mM sodium phosphate, 150 mM NaCl, 0.05% Tween 20, pH 7.4). Washes were conducted using an automatic plate washer (AccuWash, Thermo Fisher Scientific, Waltham, Mass., USA). 100 μL of NanoGlo buffer (Promega, Madison, Wisc., USA) containing 1 μL of NANOGLO® substrate (Promega, Madison, Wisc., USA) was added to each well. Following a 3 minute wait period, the signal output of each sample as relative light units (RLU) was determined using a GloMax Navigator (Promega, Madison, Wisc., USA). Signal over background (SB) was calculated by dividing the RLU from each sample from the RLU observed in the PBS control for that test (Table 3).

TABLE 3A
Titration of NANOLUC ® on Coated Plates

| | Coating Conditions | | | | | | | |
|---|---|---|---|---|---|---|---|---|
| | No Wash | | Washed | | | | | |
| | NanoLuc | NanoLuc | Mouse Anti- | Mouse Anti- | Rabbit | Rabbit | Goat | Goat |
| | Input only | Input only | NanoLuc | NanoLuc | AB | AB | AB | AB |
| MsxNanoLuc | 0 | 0 | 0.1 | 0.1 | 0.1 | 0.1 | 0.1 | 0.1 |
| Antibody | | | | | | | | |
| (ug/well) | | | | | | | | |
| Plate | Medium | Medium | Medium | Medium | Medium | Medium | Medium | Medium |
| | RLU | S/B | RLU | S/B | RLU | S/B | RLU | S/B |
| PBS | 32 | 1 | 31 | 1 | 32 | 1 | 28 | 1 |
| 3 × 10³ | 32 | 1 | 39 | 1.3 | 20 | 0.6 | 26 | 0.9 |
| mol/well | | | | | | | | |
| 3 × 10⁴ | 32 | 1 | 27 | 1.3 | 24 | 0.6 | 30 | 0.9 |
| mol/well | | | | | | | | |
| 3 × 10⁵ | 289 | 9 | 44 | 1.4 | 41 | 1.3 | 64 | 2.3 |
| mol/well | | | | | | | | |
| 3 × 10⁶ | 2693 | 84.2 | 101 | 3.3 | 198 | 6.2 | 312 | 11.1 |
| mol/well | | | | | | | | |
| 3 × 10⁷ | 32027 | 1000.8 | 1025 | 33.1 | 1661 | 51.9 | 4336 | 154.9 |
| mol/well | | | | | | | | |
| 3 × 10⁸ | 361334 | 11291.7 | 7989 | 257.7 | 18328 | 572.8 | 47652 | 1701.9 |
| mol/well | | | | | | | | |
| 3 × 10⁹ | 4212035 | 131626.1 | 85459 | 2756.7 | 239180 | 7474.4 | 515210 | 18400.4 |
| mol/well | | | | | | | | |

TABLE 3B
Titration of NANOLUC ® on Coated Plates

| | Coating Conditions | | | | | | | |
|---|---|---|---|---|---|---|---|---|
| | No Wash | | Washed | | | | | |
| | 5% BSA | 5% BSA | Mouse Anti- | Mouse Anti- | Rabbit | Rabbit | Goat | Goat |
| | Blocked | Blocked | NanoLuc | NanoLuc | AB | AB | AB | AB |
| MsxNanoLuc | 0 | 0 | 0.1 | 0.1 | 0.1 | 0.1 | 0.1 | 0.1 |
| Antibody | | | | | | | | |
| (ug/well) | | | | | | | | |
| Plate | High | High | High | High | High | High | High | High |
| | RLU | S/B | RLU | S/B | RLU | S/B | RLU | S/B |
| PBS | 39 | 1 | 32 | 1 | 32 | 1 | 100 | 1 |
| 3 × 10³ | 29 | 0.7 | 32 | 1 | 58 | 1.8 | 34 | 0.3 |
| mol/well | | | | | | | | |
| 3 × 10⁴ | 32 | 0.7 | 31 | 1 | 33 | 1.8 | 25 | 0.3 |
| mol/well | | | | | | | | |
| 3 × 10⁵ | 29 | 0.7 | 28 | 0.9 | 46 | 1.4 | 68 | 0.7 |
| mol/well | | | | | | | | |
| 3 × 10⁶ | 28 | 0.7 | 90 | 2.8 | 241 | 7.5 | 319 | 3.2 |
| mol/well | | | | | | | | |
| 3 × 10⁷ | 36 | 0.9 | 694 | 21.7 | 2234 | 69.8 | 12232 | 122.3 |
| mol/well | | | | | | | | |
| 3 × 10⁸ | 78 | 2 | 6813 | 212.9 | 18033 | 563.5 | 105519 | 1055.2 |
| mol/well | | | | | | | | |
| 3 × 10⁹ | 577 | 14.8 | 78499 | 2453.1 | 1352697 | 42271.8 | 1673864 | 16738.6 |
| mol/well | | | | | | | | |

Tables 3A-3B demonstrate that the plates coated with rabbit anti mouse IgG or goat anti mouse provided an improved orientation of the mouse anti-NANOLUC® for improved capture/binding surface. In fact, the plates coated with rabbit anti mouse IgG or goat anti mouse IgG provides higher availability for binding an indicator protein product. The indirect coating of the plates exhibited improved signal detection, which may be due to the orientation of the mouse anti-nanoluc luciferase and the availability of the binding sites to the indicator protein.

### Example 6. Detection of E. coli 0157:H7 in Whole Blood

E. coli 0157:H7 (ATCC 43888) was grown overnight in Tryptic Soy Broth with 0.05% sodium polyanethole sulfonate (TSB/SPS). Overnight growth was diluted into fresh TSB/SPS and grown to log phase (OD600 of 0.2). Log phase cells were diluted to 20000, 8000, 4000, 2000, 400, 200 and 80 CFU/mL. 200 μL of each dilution was added directly to 400 μL TSB/SPS with 200 μL, human blood to give final concentration of 5000, 2000, 1000, 500, 100, 50, and 20 CFU/mL. Human blood was collected from a single donor using sodium citrate as an anti-coagulant. A TSB positive control was made with 200 μL of each dilution added directly to 600 μL TSB to give a final concentration of 5000, 2000, 1000, 500, 100, 50, and 20 CFU/mL. After incubating the inoculated TSB and TSB/SPS/blood samples at 37° C. for two hours, 100 μL, of each dilution was transferred to a well. 10 μL, of phage CBA120.NL phage at 1.2×10⁷ plaque forming units per mL was added to each well. Assay strips were sealed with cover film and Infection was allowed to go three hours at 37° C. Following infection, these strips were washed three times with 300 μL PBS-T (10 mM sodium phosphate, 150 mM NaCl, 0.05% Tween 20, pH 7.4), TSB control was not washed. Washes were conducted using an automatic plate washer (AccuWash, Thermo Fisher Scientific, Waltham, Mass., USA). 100 μL of NanoGlo buffer (Promega, Madison, Wisc., USA) containing 1 μL of NanoGlo substrate (Promega, Madison, Wisc., USA) was added to each well. Following a 3 minute wait period, the signal output of each sample as relative light units (RLU) was determined using a GloMax Navigator (Promega, Madison, Wisc., USA). Signal over background (SB) was calculated by dividing the RLU from each sample from the RLU observed in the media control for that test (Table 4).

TABLE 4
Detection of E. coli O157:H7 in whole blood

| | | | | Washed | | | | | | | | | | | |
|---|---|---|---|---|---|---|---|---|---|---|---|---|---|---|---|
| | | | | | | | | Medium | | Medium | | High | | High | |
| | | No Wash | | | | | | Rtx Ms | | GtxMs | | Rtx Ms | | GtxMs | |
| | Initial | Medium | | Medium | | Medium | | IgG/ | | IgG/ | | IgG/ | | IgG/ | |
| Target | Cell | TSB | | BSA | | Msx | | Msx | | Msx | | Msx | | Msx | |
| Cell/ml | 100 ul | Control | S/B | Blocked | S/B | Nanoluc | S/B | Nanoluc | S/B | Nanoluc | S/B | Nanoluc | S/B | Nanoluc | S/B |
| 0 | 0 | 53 | 1 | 15 | 1 | 24 | 1 | 21 | 1 | 15 | 1 | 14 | 1 | 21 | 1 |
| 20 | 2 | 7383 | 139 | 18 | 1 | 2148 | 90 | 61 | 3 | 31 | 2 | 14 | 1 | 176 | 8 |
| 50 | 5 | 53412 | 1008 | 21 | 1 | 2171 | 90 | 2624 | 125 | 14 | 1 | 17646 | 1260 | 7813 | 372 |
| 100 | 10 | 159201 | 3004 | 31 | 2 | 9395 | 391 | 3423 | 163 | 2929 | 195 | 2070 | 148 | 10414 | 496 |
| 500 | 50 | 644322 | 12157 | 33 | 2 | 7183 | 299 | 6306 | 300 | 31656 | 2110 | 19567 | 1398 | 21157 | 1007 |
| 1000 | 100 | 1771798 | 33430 | 57 | 4 | 7550 | 315 | 25984 | 1237 | 33686 | 2246 | 21298 | 1521 | 46320 | 2206 |
| 2000 | 200 | 4401842 | 83054 | 281 | 19 | 54376 | 2266 | 40387 | 1923 | 58394 | 3893 | 46128 | 3295 | 108382 | 5161 |
| 5000 | 500 | 12730663 | 240201 | 181 | 12 | 134728 | 5614 | 167273 | 7965 | 253412 | 16894 | 168783 | 12056 | 213343 | 10159 |

### Example 7. Peptide-Tagged Indicator Phage Detection of Bacteria using a Lateral Flow Strip/Dipstick

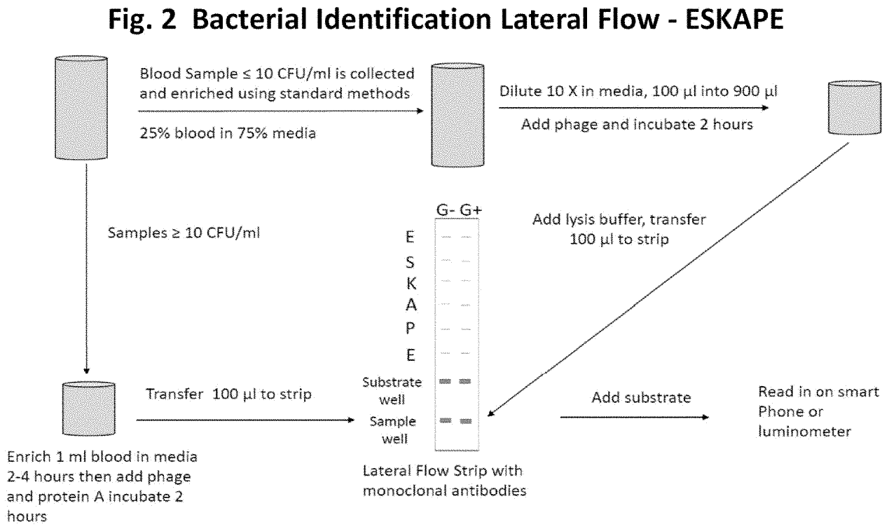

As shown in FIG. 2, a 1 mL sample of 25% whole blood culture in 75% media (e.g., sodium polyanethol sulfonate) is enriched for 5-6 hours. A multiplex phage cocktail (including, for example, indicator phage cocktails for each of the following: Pseudomonas phage, Enterobacter phage, Enterococcus phage, Klebsiella phage, S. aureus phage, and A. baumannii phage) is added to the enriched blood and incubated for 2 hours. To permit infection in wells containing blood matrix, 0.5 mg of recombinant Staphylococcal protein A (pro-356, Prospec, Ness-Ziona, Israel) per well is included within the 20 μL of multiplex phage cocktail working stock. Each indicator phage cocktail comprises bacteriophage with a unique capture tag (i.e., Pseudomonas phage cocktail has tag A, Enterobacter phage cocktail has tag B, Enterococcus phage cocktail has tag C, Klebsiella phage cocktail has tag D, S. aureus phage cocktail has tag E, and A. baumannii phage cocktail has tag F. After 3 hours of incubation, lysis buffer is added. 100 μL of the lysed recombinant bacteriophage sample is loaded onto a lateral flow strip coated with binding partners specific for each of the unique capture tags. Substrate is then added to the lateral flow strip and the strip is read using a handheld luminometer.

### Example 8. Peptide-Tagged Indicator Phage Detection of Bacteria using a Microtiter Plate

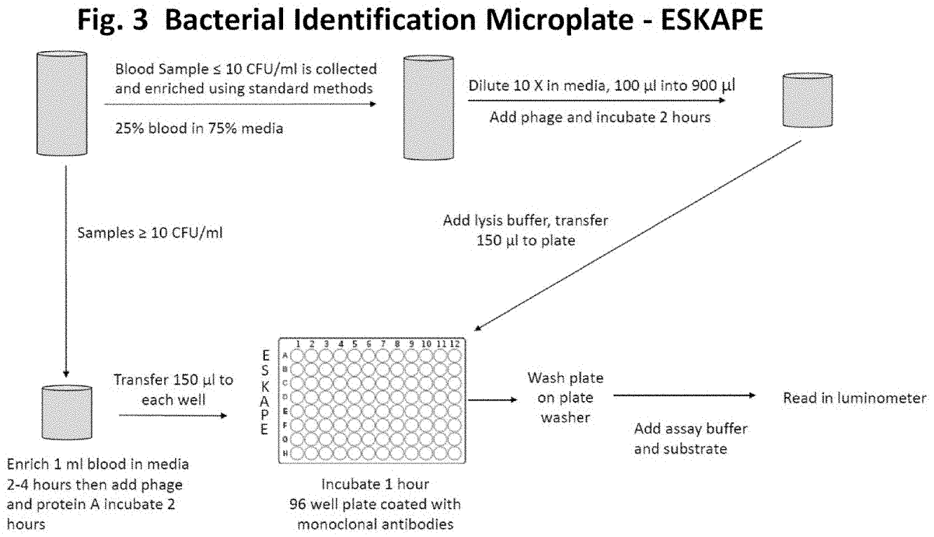

As shown in FIG. 3, a 1 mL sample of 25% whole blood culture in 75% media (e.g., sodium polyanethol sulfonate) is enriched for 5-6 hours. A multiplex phage cocktail (including, for example, indicator phage cocktails for each of the following: Pseudomonas phage, Enterobacter phage, Enterococcus phage, Klebsiella phage, S. aureus phage, and A. baumannii phage) is added to the enriched blood and incubated for 2 hours. To permit infection in wells containing blood matrix, 0.5 mg of recombinant Staphylococcal protein A (pro-356, Prospec, Ness-Ziona, Israel) per well is included within the 20 μL of multiplex phage cocktail working stock. Each indicator phage cocktail comprises bacteriophage with a unique capture tag (i.e., Pseudomonas phage cocktail has tag A, Enterobacter phage cocktail has tag B, Enterococcus phage cocktail has tag C, Klebsiella phage cocktail has tag D, S. aureus phage cocktail has tag E, and A. baumannii phage cocktail has tag F). After 2 hours of incubation, lysis buffer is added. 100 μL of the lysed recombinant bacteriophage sample is loaded into each well of the microtiter plate coated with binding partners specific for each of the unique capture tags. Each well is tagged with a different monoclonal antibody. For example, if the Enterobacter-specific bacteriophages are tagged with Tag B, the wells coated with mouse anti-Tag B would be capable of capturing the indicator protein The sample incubates for 60 mins and then the pate is washed. Substrate is then added to each well and luminescence is measured using a luminometer.

### Example 9. Capture of HA-tagged Nanoluc from E. coli 0157:H7

A peptide-tagged indicator, HA (hemagglutinin)-tagged NANOLUC, added to the bacteriophage JGO4 genome, was used in capture experiments. Human influenza hemagglutinin is a peptide of 9 amino acids with sequence of YPYDVPDYA. The HA tag was fused to the C-terminus of Nanoluc (a linker, GSSG, was put between Nanoluc and HA), the recombinant phage JG04.NL-HA was made and isolated. After infection of E. coli 0157:H7, Nanoluc-HA was expressed during phage replication. Plates coated with a rabbit anti-HA were used to capture the expressed Nanoluc-HA, with both infection and capture occurring during the 3 hour infection. Substrate was added after washing of the wells with PBS/Tween.

### Capture Plates

To prepare capture plates, RbxHA antibody (Invitrogen, Catalog # 71-5500) was diluted in PBS (Sigma PBS (pH 7.6) Catalog #P3744) to 1 μg/mL or 10 μg/mL. Diluted antibody (100 μL) or PBS (negative control) was transferred to each well of Greiner Bio-One Strip Plates, Catalog #762075 (Lumitrac—white medium binding), 4 strips per dilution (0.1 μg/well or 1.0 μg/well). Plates were sealed with film and stored at 4° C. overnight. Antibody was aspirated and plates were washed once with 300 L of PBS. Blocking solution (300 μL) of 5% BSA in PBS (Sigma BSA, Catalog #A9647) was added to each well and plates were sealed with film and incubated for 2 h at room temperature. Blocking solution was aspirated and wells washed once with 300 μL of PBS. Overcoat solution (300 μL) of 5% sucrose in PBS (Fisher BioReagents D-Sucrose, Catalog #BP220-1) was added to each well. Plates were sealed with film and incubated 1 h at room temperature. Overcoat solution was aspirated overcoat and plates were inverted and left to dry overnight at room temperature. The following day plates were stored in sealed bag at 4° C. until use.

### Tagged Nanoluc Capture—Assay Protocol

An overnight culture of E. coli 0157:H7 (ATCC 43888) was diluted in TSB to 1000 and 100000 CFU/mL. Bacterial dilutions or TSB (100 μL, no bacteria control) to each well. Both coated capture strips and uncoated strips were used for comparison. Phage working stock was added (10 μL of 1.1×10⁷ pfu/mL in SM Buffer). Plates were sealed with film and incubated for 3 h for infection at 37° C. The sample was removed and wells were washed five times with 300 μL of 1X PBS-T (Seracare 10 X PBS-Tween, Catalog #5460-0027). Detection Master Mix (100 μL) was added to each well (50 μL of NanoGlo Buffer, Promega Catalog #N112B, 50 μL of PBS, and 1 μL of NanoGlo substrate, Promega Catalog #N113B). Plates were read on GloMax Navigator (3 min delay, 1 s integration, two reads).

### Capture of HA-Nanoluc from E. coli 0157:117

Stationary phase E. coli 0157:H7 ATCC 43888 were diluted to 100000 and 1000 CFU/mL, 100 μL used per well and infected for 3 hours with indicator recombinant phage. JG04.NL does not include the HA tag and JG04.NL.NA includes the tag. No consistent difference in signal was observed from either phage using uncoated/unwashed strips or PBS-coated washed strips. Consistently higher signal was observed with tagged JG04.NL-HA using the anti-HA capture strips, a 10-fold increase over untagged at the 10,000 CFU burden. Non-specific binding of Nanoluc results in signal background of approximately 1,000 RLU at 10000 CFU and <100 RLU for 100 CFU in blocked plates. Plated CFU and RLU results are shown in Tables 5 and 6 below and represent an average of replicate wells.

TABLE 5
Capture of HA-Nanoluc from E. coli O157:H7 -
0.1 μg per well antibody coating

| | | | | |
|---|---|---|---|---|
| Target | Plated | JG04.NL | JG04.NL-HA | Ratio of |
| CFU | CFU | RLU | RLU | Tag/Untag |

Uncoated - Unblocked - Unwashed

| | | | | |
|---|---|---|---|---|
| 10,000 | N/T | 1754000 | 1953667 | 1.1 |
| 100 | 71.5 | 2142 | 2339 | 1.1 |
| 0 | N/T | 27 | 24 | 0.9 |

PBS coated - Blocked -Washed

| | | | | |
|---|---|---|---|---|
| 10,000 | N/T | 1001 | 897 | 0.9 |
| 100 | 71.5 | 61 | 6 | 0.1 |
| 0 | N/T | 6 | 5 | 0.8 |

0.1 μg Anti-HA coated - Blocked -Washed

| | | | | |
|---|---|---|---|---|
| 10,000 | N/T | 3260 | 35997 | 11.0 |
| 100 | 71.5 | 14 | 130 | 9.1 |
| 0 | N/T | 11 | 6 | 0.6 |

TABLE 6
HA Tag Capture - 1 μg per well antibody coating

| | | | |
|---|---|---|---|
| Target | JG04.NL | JG04.NL-HA | Ratio of |
| CFU | RLU | RLU | Tag/Untag |

Uncoated - Unblocked - Unwashed

| | | | |
|---|---|---|---|
| 10,000 | 2335500 | 2387000 | 1.0 |

PBS coated - Blocked - Washed

| | | | |
|---|---|---|---|
| 10,000 | 834 | 486 | 0.6 |

0.1 μg anti-HA coated - Blocked - Washed

| | | | |
|---|---|---|---|
| 10,000 | 2253 | 19670 | 8.7 |

1 μg anti-HA coated - Blocked - Washed

| | | | |
|---|---|---|---|
| 10,000 | 1999 | 89845 | 44.9 |

We claim:

1. A recombinant bacteriophage comprising a genetic construct inserted into the bacteriophage genome, wherein the genetic construct comprises an indicator gene and a capture moiety.

2. The bacteriophage of claim 1, wherein the genetic construct further comprises an exogenous promoter.

3. The bacteriophage of claim 1, wherein the indicator gene is a luciferase, horseradish peroxidase, or an alkaline phosphatase.

4. The bacteriophage of claim 1, wherein the indicator gene and capture moiety encode a capture moiety-indicator protein fusion product.

5. The bacteriophage of claim 1, wherein the capture moiety is a peptide or protein tag.

6. An indicator cocktail composition comprising at least two different recombinant bacteriophages, wherein the recombinant bacteriophages each comprise a genetic construct according to claim 1.

7. The cocktail composition of claim 6, wherein the at least two different recombinant bacteriophages are specific to the same bacteria of interest.

8. A multiplex cocktail composition comprising at least two indicator cocktail compositions according to claim 6, wherein the first indicator cocktail composition is specific for a first bacteria of interest and the second cocktail composition is specific for a second bacteria of interest.

9. A method of detecting a bacteria of interest in a sample comprising the steps of:
(i) obtaining the sample;
(ii) incubating the sample with at least one indicator cocktail composition, wherein the recombinant bacteriophage comprises a genetic construct inserted into the bacteriophage genome, and wherein the genetic construct comprises an indicator gene encoding an indicator protein product;
(iii) capturing the indicator protein product; and
(iv) detecting the captured indicator protein product, wherein detection of the captured indicator protein product indicates the bacteria of interest is present in the sample.

10. A method of detecting a bacteria of interest in a sample comprising the steps of:
(i) obtaining the sample;
(ii) incubating the sample with at least one indicator cocktail composition, wherein the recombinant bacteriophage comprises a genetic construct inserted into the bacteriophage genome, and wherein the genetic construct comprises an indicator gene and a capture moiety encoding a capture moiety-indicator protein fusion product;
(iii) capturing the capture moiety-indicator protein fusion product; and
(iv) detecting the captured indicator protein product, wherein detection of the captured indicator protein product indicates the bacteria of interest is present in the sample.

11. The method of claim 9 or 10, wherein the bacteria of interest is a genus, species, strain, or type of bacteria.

12. The method of claim 9 or 10, wherein the genetic construct further comprises an exogenous promoter.

13. The method of claim 9 or 10, wherein the indicator gene encodes an enzyme.

14. The method of claim 13, wherein the enzyme is a luciferase or an alkaline phosphatase.

15. The method of claim 9 or 10, wherein the indicator cocktail composition comprises at least two recombinant bacteriophages specific for the same bacteria of interest.

16. The method of claim 10, further comprising incubating the sample with at least two indicator cocktail compositions, wherein a first indicator cocktail composition comprises bacteriophages specific for a first bacteria of interest and a second indicator cocktail composition comprises bacteriophages specific for a second bacteria of interest.

17. The method of claim 16, wherein the bacteriophages of the first indicator cocktail composition each comprise a first capture moiety and the bacteriophages of the second indicator cocktail composition comprise a second capture moiety.

18. The method of claim 10, wherein the capture moiety is a peptide tag.

19. The method of claim 9 or 10, further comprising a step for lysing the bacteria in the sample following incubation with the indicator cocktail composition.

20. The method of claim 9, wherein capturing the indicator protein product comprises contacting the sample with a surface comprising a binding partner capable of capturing the indicator protein product.

21. The method of claim 20, wherein the binding partner is an antibody or a fragment thereof.

22. The method of claim 10, wherein capturing the capture moiety-indicator protein fusion product comprises contacting the sample with a surface comprising a binding partner capable of capturing the capture moiety-indicator protein fusion product.

23. The method of claim 22, wherein the surface comprises a first binding partner specific for a first capture moiety and a second binding partner for a second capture moiety.

24. The method of claim 9 or 10, further comprising a step for determining the antibiotic resistance of the bacteria of interest detected in the sample by incubating the sample with an antibiotic prior to incubating the sample with the at least one indicator cocktail composition.

25. The method of claim 9 or 10, wherein the sample is a biological, food, environmental, water, or commercial sample.

26. The method of claim 25, wherein the biological sample is blood, serum, or plasma.

27. The method of claim 26, wherein protein A is added to the sample.

28. The method of claim 25, wherein the food sample comprises meat, fish, vegetables, fruit, eggs, dairy products, dried food products, or powdered infant formula.

29. The method of claim 9 or 10, wherein the bacteria of interest is at least one of Enterococcus faecium, Staphylococcus aureus, Klebsiella Pneumoniae, Acinetobacter baumanni, Pseudomonas aeruginosa, Salmonella spp Cronobacter spp., Campylobacter spp., Listeria spp., Enterobacter spp. or E. coli.

30. A kit for detecting a bacteria of interest comprising:
(i) recombinant bacteriophage comprising a genetic construct inserted into the bacteriophage genome, wherein the recombinant bacteriophage comprises a genetic construct inserted into the bacteriophage genome, and wherein the genetic construct comprises an indicator gene and a capture moiety encoding a capture moiety-indicator protein fusion product; and
(ii) a surface for capturing the capture moiety-indicator protein fusion product, wherein the surface comprises a binding partner specific for the capture moiety.

31. The kit of claim 30, further comprising at least one of: a detection reagent, a lysis buffer, a microtiter plate, latex particle, lateral flow strip, microsphere, or dipstick.

32. A system for detecting a bacteria of interest comprising:
(a) a recombinant bacteriophage comprising a genetic construct inserted into the bacteriophage genome, wherein the recombinant bacteriophage comprises a genetic construct inserted into the bacteriophage genome, and wherein the genetic construct comprises an indicator gene and a capture moiety encoding a capture moiety-indicator protein fusion product;
(b) a surface for capturing capture moiety-indicator protein fusion product, wherein the surface comprises a binding partner specific for the capture moiety; and
(c) a component for detecting the indicator protein product.
